(19) United States
(12) Patent Application Publication
(10) Pub. No.: US 2023/0293330 A1
(43) Pub. Date: Sep. 21, 2023

(54) SUSPENSION BOOT

(71) Applicant: Avera Health, Sioux Falls, SD (US)

(72) Inventors: Matthew IBARRA, Cerritos, CA (US); Nicolle SAMUELS, Harrisburg, SD (US); Benjamin DADACAY, Jr., Long Beach, CA (US); Craig OVANS, Chandler, AZ (US); Myong KIM, Tustin, CA (US)

(21) Appl. No.: 18/006,319

(22) PCT Filed: Jul. 23, 2021
(86) PCT No.: PCT/US2021/043056

§ 371 Date: Jan. 20, 2023

Related U.S. Application Data
(60) Provisional application No. 63/055,730, filed on Jul. 23, 2020.

(51) Int. Cl. A61F 5/01; A43B 7/14

(52) U.S. Cl. CPC A61F 5/0127; A43B 7/14; A61F 2005/0169; A61F 2005/0139

(57) ABSTRACT

Methods, devices, and systems are described for a foot suspension system configured to offload weight from a foot of a user. The foot suspension system includes a boot shaped to receive at least a part of the foot of the user, the boot including a bottom inner surface. The foot suspension system further includes a sleeve configured to secure to a lower appendage of the user to assist with suspending at least the part of the foot above the bottom inner surface of the boot. The foot suspension system further comprises at least one vertical bar with a first end positioned adjacent to the boot and a second end of the at least one vertical bar positioned

### CROSS-REFERENCE TO RELATED APPLICATION

This application claims priority under 35 U.S.C. § 119(a) to U.S. Provisional application Ser. No. 63/055,730, filed on Jul. 23, 2020 and entitled “OFFLOAD SYSTEM,” the disclosure of which is incorporated by reference herein in its entirety.

### TECHNICAL FIELD

The present disclosure relates generally to medical devices, and more particularly, to a suspension boot.

### BACKGROUND

Diabetic foot ulcers are a chronic open sore or wound that occurs on the bottom of the foot, at the forefoot, midfoot or hindfoot, along the top and bottom of each toe, or on the sides of the feet. A typical deterioration pattern for feet of a person suffering from diabetes follows a pattern of diabetes onset, followed by neuropathy, ulceration, infection, ischemia, and ultimately amputation of the affected foot.

Currently, total contact casts are considered the current ‘gold standard’ treatment for diabetic foot ulcers because the casts shift the weight of the user onto the cast. However, adoption rate is low due to their limited mobility, increased difficulty in walking, and high costs to patients. Total contact casts also present drawbacks at clinics and treatment centers. Total contact casts must be destroyed at every clinician visit to access the foot ulcers. Following the treatment, a new total contact cast must be reapplied. Diabetic foot ulcers treated with total contact cast costs average five to six weeks and require that new total contact casts are applied for every clinical visit for successful treatment. As such, total contact casts and their limitations lead to high costs and a poor quality of patient life.

### SUMMARY

The present disclosure provides methods, devices, systems, and articles of manufacture for a foot suspension system.

In one aspect, a foot suspension system configured to offload weight from a foot of a user is described. The foot suspension system includes a boot shaped to receive at least a part of the foot of the user, the boot including a bottom inner surface. The foot suspension system further includes a sleeve configured to secure to a lower appendage of the user to assist with suspending at least the part of the foot above the bottom inner surface of the boot. The foot suspension system further comprises at least one vertical bar including a first end and a second end, the first end of the at least one vertical bar positioned adjacent to the boot and the second end of the at least one vertical bar positioned adjacent to the sleeve, the at least one vertical bar configured to support the sleeve above the boot thereby assisting with suspending at least the part of the foot above the bottom inner surface of the boot. The foot suspension system further includes at least one rotatable pivot rotatably coupling the first end of the at least one vertical bar to the boot, the rotatable pivot allowing pivoting of the at least one vertical bar and the sleeve relative to the boot.

In some variations, the rotatable pivot further comprises a dampening mechanism configured to dampen the pivoting of the at least one vertical bar and the sleeve relative to the boot, wherein the dampening mechanism includes at least one of a magnet, an elastomeric material, a spring, a wafer spring, a honeycomb insert, and an in-mold spring. Additionally, the rotatable pivot further comprises an engagement feature coupled to the first end of the at least one vertical bar and configured to compress the dampening mechanism as the at least one vertical bar pivots relative to the boot. Further, the rotatable pivot further comprises a radial protrusion feature configured to be inserted into a cutout in the dampening mechanism, the radial protrusion feature configured to secure the dampening mechanism in place at the rotatable pivot. In some variations, the radial protrusion feature is fixed by a plurality of teeth in a circular aperture at a rear area of the boot. Additionally, the dampening mechanism includes a compressible insert configured to be fixed in place by at least the radial protrusion feature in a circular aperture.

In some variations, the rotatable pivot further comprises an engagement feature coupled to the first end of the at least one vertical bar and configured to secure the compressible insert in place and configured to compress the compressible insert as the at least one vertical bar pivots relative to the boot. Further, the engagement feature is rotatable to the boot thereby compressing the compressible insert. In some variations, the cutout is in the compressible insert and the cutout is offset from a center of the compressible insert thereby enabling a greater dampening in a forward pivoting direction of the at least one vertical bar than a backward pivoting direction of the at least one vertical bar. Additionally, the cutout is in the compressible insert and the cutout is offset from a center of the compressible insert thereby enabling a greater dampening in a backward pivoting direction of the at least one vertical bar than a forward pivoting direction of the at least one vertical bar.

In some variations, the foot suspension system further comprises a first gear including a plurality of teeth extending around a circumference of the first gear and a second gear configured to couple to the plurality of teeth of the first gear, the second gear configured to rotate the first gear for adjusting an angle of the at least one vertical bar with respect to the boot. Additionally, the first gear includes the radial protrusion feature and wherein the second gear is a worm gear having an exposed end configured to rotate the worm gear.

In another aspect, a foot suspension system configured to offload weight from a foot of a user is described. The foot suspension system includes a boot shaped to receive at least a part of the foot of the user, the boot including a bottom inner surface. The foot suspension system further includes a sleeve configured to secure to a lower appendage of the user to assist with suspending at least the part of the foot above the bottom inner surface of the boot. The foot suspension system further comprises at least one vertical bar including a first end and a second end, the first end of the at least one vertical bar positioned adjacent to the boot and the second end of the at least one vertical bar positioned adjacent to the sleeve, the at least one vertical bar configured to support the sleeve above the boot thereby assisting with suspending at least the part of the foot above the bottom inner surface of the boot. The foot suspension system further includes a cushioning mechanism coupled to the at least one vertical bar and configured to absorb a downward force applied to the sleeve.

In some variations, the cushioning mechanism is at least one of a magnet, an elastomeric material, a spring, a wafer spring, a honeycomb insert, and an in-mold spring. The cushioning mechanism is configured to be angled at an oblong aperture at the at least one vertical bar, the at least one vertical bar configured to move downward in response to force applied at the sleeve. In some variations, the cushioning mechanism is configured to be inserted into an aperture at a front portion of the at least one vertical bar, the cushioning mechanism configured to dampen movement of the at least one vertical bar with respect to the boot, wherein the front portion of the at least one vertical bar covers a front side of the sleeve.

Additionally, the cushioning mechanism is configured to be inserted into an aperture at a back portion of the at least one vertical bar, the cushioning mechanism configured to dampen movement of the at least one vertical bar with respect to the boot, wherein the back portion of the at least one vertical bar covers a back side of the sleeve. Further, the at least one vertical bar further comprises a hinge configured to pivot the at least one vertical bar in a direction away from the boot and configured to extend the at least one vertical bar at an angle away from the boot. Additionally, the hinge further configured to pivot the at least one vertical bar in the direction away from the boot in response to the sleeve receiving a user appendage. In some variations, the foot suspension system includes a vertically-oriented spring attached to a rotatable pivot and configured to absorb the downward force applied to the sleeve.

In yet another aspect, a foot suspension system configured to offload weight from a foot of a user is described. The foot suspension system includes a boot shaped to receive at least a part of the foot of the user, the boot including a bottom inner surface. The foot suspension system further includes a sleeve configured to secure to a lower appendage of the user to assist with suspending at least the part of the foot above the bottom inner surface of the boot. The foot suspension system further comprises at least one vertical bar including a first end and a second end, the first end of the at least one vertical bar positioned adjacent to the boot and the second end of the at least one vertical bar positioned adjacent to the sleeve, the at least one vertical bar configured to support the sleeve above the boot thereby assisting with suspending at least the part of the foot above the bottom inner surface of the boot.

In some variations, the at least one vertical bar is adjustable to selectively secure the sleeve at more than one distance relative to the boot. Additionally, the at least one vertical bar further comprises a shaft configured to adjust a height of the at least one vertical bar via a stepping mechanism. Further, the shaft includes an elongated aperture in a mid-section of the shaft through which a screw is inserted for securing the shaft to the at least one vertical bar. In some variations, the shaft includes a corrugated surface for immobilizing the shaft against the at least one vertical bar.

In some variations, the foot suspension system includes a dorsal foot covering configured to cover a toe area of the boot and attach to a rear area of the boot, the dorsal foot covering configured to pivot to access the toe area of the boot, the dorsal foot covering configured to selectively attach to a front lip of the toe area. Additionally, the dorsal foot covering is configured to selectively detach from the rear area of the boot for accessing the foot of the user. Further, the foot suspension system includes a strap configured to wrap around the dorsal foot covering and coupled to a rotatable pivot. In some variations, the foot suspension system includes a strap configured to wrap around the dorsal foot covering and configured to attach to a backside of the boot.

In some variations, the foot suspension system includes at least one sleeve anchor having an aperture for receiving the at least one vertical bar, the at least one sleeve anchor configured to selectively detach from the at least one vertical bar for decoupling the sleeve from the at least one vertical bar. In some variations, a protrusion configured to be inserted an opening at the at least one vertical bar for securing the at least one sleeve anchor to the at least one vertical bar. In some variations, the sleeve anchor further comprises: a plurality of holes for fastening the sleeve anchor to a sleeve harness having a loop for securing the sleeve to the sleeve anchor.

In some variations, the foot suspension system comprises a sleeve harness configured to secure the sleeve to the at least one vertical bar, the sleeve harness including a loop configured to secure a horizontal strap wrapped around the sleeve to a sleeve anchor. In some variations, the sleeve harness includes at least two vertical straps for securing the horizontal strap. In some variations, the sleeve harness includes a clamp configured to extend around a circumference of the sleeve. In some variations, the at least one vertical bar wraps around a front portion of the sleeve, the at least one vertical bar configured to cover the front portion of the sleeve. Additionally, the at least one vertical bar wraps around a back portion of the sleeve, the at least one vertical bar configured to cover the back portion of the sleeve. Additionally, the boot includes a cutout for ventilation. Further, a boot sidewall flexes at a midpoint in response to a movement by the sleeve with respect to the boot. Additionally, the bottom inner surface is sloped downward from a rear area of the boot to a toe area of the boot.

In yet another aspect, a method is described. The method includes receiving at least a part of a foot of a user in a boot of a foot suspension system, the foot suspension system comprising a foot suspension system that may include a boot shaped to receive at least a part of the foot of the user, the boot including a bottom inner surface. The foot suspension system further includes a sleeve configured to secure to a lower appendage of the user to assist with suspending at least the part of the foot above the bottom inner surface of the boot. The foot suspension system further comprises at least one vertical bar including a first end and a second end, the first end of the at least one vertical bar positioned adjacent to the boot and the second end of the at least one vertical bar positioned adjacent to the sleeve, the at least one vertical bar configured to support the sleeve above the boot thereby assisting with suspending at least the part of the foot above the bottom inner surface of the boot. The method may include securing the sleeve of the foot suspension system to a lower extremity of the user to cause a bottom of the foot to suspend a first distance above an inner surface of the boot at least when a walking force is applied to the sleeve.

In some variations, the foot suspension system may further include at least one rotatable pivot rotatably coupling the first end of the at least one vertical bar to the boot, the at least one rotatable pivot allowing pivoting of the at least one vertical bar and the sleeve relative to the boot. In some variations, the foot suspension system may further include a dampening mechanism coupled to the at least one vertical bar and configured to absorb a downward force applied to the sleeve. In some variations, the dampening mechanism is configured to be angled at an oblong aperture at the at least one vertical bar, the at least one vertical bar configured to move downward in response to force applied at the sleeve. In some variations, the at least one vertical bar is adjustable to selectively secure the sleeve at more than one distance relative to the boot.

In some variations, the foot suspension system may further comprise at least one sleeve anchor having an aperture for receiving the at least one vertical bar, the sleeve anchor configured to selectively detach from the at least one vertical bar for decoupling the sleeve from the at least one vertical bar. In some variations, the foot suspension system may further comprise a sleeve harness configured to secure the sleeve, the sleeve harness including loops configured to secure horizontal straps wrapped around the sleeve to a sleeve anchor. In some variations, the at least one vertical bar may further comprise a hinge configured to rotate the at least one vertical bar in an outward direction away from the boot for adjusting to a size of a user calf. In some variations, the foot suspension system may further comprise a dorsal foot covering configured to cover a toe area of the boot and attach to a rear area of the boot, the dorsal foot covering configured to pivot to access the toe area of the boot, the dorsal foot covering configured to selectively attach to a front lip of the toe area. In some variations, the dorsal foot covering is configured to selectively detach from the rear area of the boot for accessing the foot of the user.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes, it should be readily understood that such features are not intended to be limiting. The claims that follow this disclosure are intended to define the scope of the protected subject matter.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1A depicts a side view of a foot suspension system having a vertical bar configured to support a sleeve above the boot;

FIG. 1B depicts a side perspective view of a foot suspended in a foot suspension system;

FIG. 2 depicts a side perspective view of a foot suspension system having a rotatable pivot;

FIG. 3 depicts a perspective side view of another embodiment of a foot suspension system having a rotatable pivot;

FIG. 4 depicts an exploded view of a rotatable pivot;

FIG. 5A depicts a configuration of a compression insert in a rotatable pivot;

FIG. 5B depicts another configuration of a compression insert in a rotatable pivot;

FIG. 5C depicts yet another configuration of a compression insert in a rotatable pivot;

FIG. 5D depicts yet another configuration of a dampening mechanism in a rotatable pivot;

FIG. 5E depicts another exploded view of a rotatable pivot configured to interface with the embodiment of FIG. 5D;

FIG. 6A depicts an exploded view of a gear for adjusting an angle of the vertical bar with respect to the boot;

FIG. 6B depicts an assembled view of a gear for adjusting an angle of the vertical bar with respect to the boot;

FIG. 7A depicts a side perspective view of another embodiment of the foot suspension system having a vertical bar configured to absorb a downward force applied to the sleeve;

FIG. 7B depicts a side perspective view of another embodiment of the foot suspension system having a vertical bar configured to absorb a downward force applied to the sleeve;

FIG. 8A depicts a side perspective view of a foot suspension system having a dampening mechanism inserted into an aperture at a front portion and a back portion of the vertical bar;

FIG. 8B depicts a side perspective view of an embodiment of the boot of FIG. 8A having another dampening mechanism inserted into an aperture at a front portion and a back portion of the vertical bar;

FIG. 8C depicts a side perspective view of a foot suspension system having a lacing feature;

FIG. 9A depicts a side perspective view of an embodiment of the boot having a hinge configured to pivot the vertical bar in a direction away from the boot;

FIG. 9B depicts a side perspective view of an embodiment of the boot of FIG. 9A having a hinge outwardly rotated away from the boot;

FIG. 10 depicts a side perspective view of an embodiment of the foot suspension system including a vertically oriented spring configured to absorb the downward force applied by the sleeve;

FIG. 11A depicts an exploded view of an embodiment of an adjustable vertical bar having a shaft and a corrugated surface;

FIG. 11B depicts an assembled side perspective view of the adjustable vertical bar of FIG. 11A having a shaft and a corrugated surface;

FIG. 12 depicts a side view of a dorsal foot covering configured to pivot to access the toe area of the boot;

FIG. 13 depicts a side perspective view of another embodiment of the boot having a strap configured to wrap around the dorsal foot covering and coupled to a rotatable pivot;

FIG. 14 depicts a back perspective view of another embodiment of the boot having a strap configured to wrap around the dorsal foot covering and configured to attach to a backside of the boot;

FIG. 15A depicts an exploded view of an embodiment of a foot suspension system having a sleeve anchor with an aperture for receiving the vertical bar;

FIG. 15B depicts an exploded view of another embodiment of the foot suspension system having a sleeve anchor configured to couple with a sleeve harness;

FIG. 15C depicts a side view of another embodiment of the foot suspension system having another sleeve anchor for receiving the vertical bar;

FIG. 15D depicts a side view of another embodiment of the foot suspension system having another sleeve anchor for receiving the vertical bar;

FIG. 15E depicts a side view of a sleeve anchor assembled to the sleeve harness with the horizontal securement straps;

FIG. 15F depicts a side view of a locking mechanism assembled to the sleeve harness;

FIG. 16 depicts an exploded view of another embodiment of the foot suspension system having a clamp configured to extend around a circumference of the sleeve;

FIG. 17A depicts an exploded view of another embodiment of the foot suspension system having a front vertical bar and a rear vertical bar wrapped around a front sleeve portion and a rear sleeve portion;

FIG. 17B depicts an assembled view of the foot suspension system of FIG. 17A having a front vertical bar and a rear vertical bar wrapped around a front sleeve portion and a rear sleeve portion;

FIG. 18 depicts a perspective side view of a foot suspension system having an embodiment of the boot sidewall configured to flex at a midpoint;

FIG. 19 depicts a perspective side view of an inner boot cover;

FIG. 20A depicts a perspective side view of an inner sleeve having a slip guard; and

FIG. 20B depicts a perspective side view of an inner sleeve having a cinching slip guard.

### DETAILED DESCRIPTION

A suspension boot may allow a user to offload weight from an injured foot, such as a foot having chronic wounds, foot ulcers, Charcot foot, or post-surgical incisions. The suspension boot may protect the injured foot while allowing user mobility, such as walking. For example, the suspension boot may allow the user to walk using the injured foot while preventing harm to existing foot injuries (e.g., foot ulcers) and/or formation of new injuries (e.g., additional foot ulcers and/or worsening of foot health). The suspension boot can be configured to shift the weight of the user to the suspension boot rather than the foot of the user, such as during walking. The suspension boot may be used as an orthopedic device, prosthetic, or ankle foot orthosis. Types of ankle foot orthoses may include external orthopedic appliances, braces, or splints that are devices to control, limit, or assist foot and ankle motion and provide leg support.

In some embodiments, the suspension boot may include a boot, a sleeve, and a vertical bar. The boot may be configured to receive a user foot such that the foot is at least partially contained by the boot. The boot may include a bottom surface over which the user foot maintains a predetermined clearance. The sleeve may be configured to secure to a part of a lower appendage (e.g., calf, lower leg) of a user to assist with suspending the user foot above the inner surface of the boot. The vertical bar of the suspension boot may be configured to extend between and couple to the boot and the sleeve. The vertical bar may be configured to support the sleeve above the boot thereby supporting the foot of the lower appendage secured in the sleeve above the inner surface of the boot.

The suspension boot embodiments described herein may improve one or more problems associated with other boot devices for users. For example, the suspension boot may improve user mobility, improve user ease of walking, improve user comfort, and lower costs to patients suffering from a foot injury (e.g., diabetic foot ulcers). The suspension boot may include a variety of features for assisting with providing such improvements. For example, some embodiments of the suspension boot can include a rotatable pivot and a dampening mechanism to enable a natural walk and reduce pain. Additionally, in some embodiments, the suspension boot may include a gear to adjust the angle of the sleeve and vertical bar relative to the boot for preventing foot soreness and fatigue. The suspension boot may include an adjustable vertical bar configured to adjust a distance between the sleeve and the bottom surface of the boot based on leg length and size. Further, in some embodiments, the suspension boot may be configured to absorb a downward force applied to the sleeve thereby offloading the downward force away from the injured foot and allowing the injured foot to heal. The suspension boot enhances user comfort with ventilation and a detachable sleeve.

Additionally, the suspension boot embodiments described herein may improve one or more problems associated with other boot devices for clinicians. For example, the suspension boot may improve a clinician's access to the foot of the user. In some embodiments, the suspension boot includes a sleeve anchor configured to releasably detach the sleeve from the vertical bar and the boot. Additionally, the suspension boot may include a selectively detachable dorsal cover that enables easy access to the foot. This is a significant advantage over total contact casts which must be broken to access the foot for each clinician visit. Furthermore, the adjustable vertical bar may enable clinicians to prescribe a particular distance between the user foot and the bottom inner surface of the boot. As the foot recovers, the clinician may prescribe a smaller distance between the user foot and the bottom inner surface of the boot. In some instances, the clinician may prescribe partial contact between the user foot and the bottom inner surface of the boot.

The methods, systems, and apparatuses described herein are for a foot suspension system configured to offload a user weight. The various embodiments also disclose a suspension boot configured to maximize comfort, access to the foot, and natural walking.

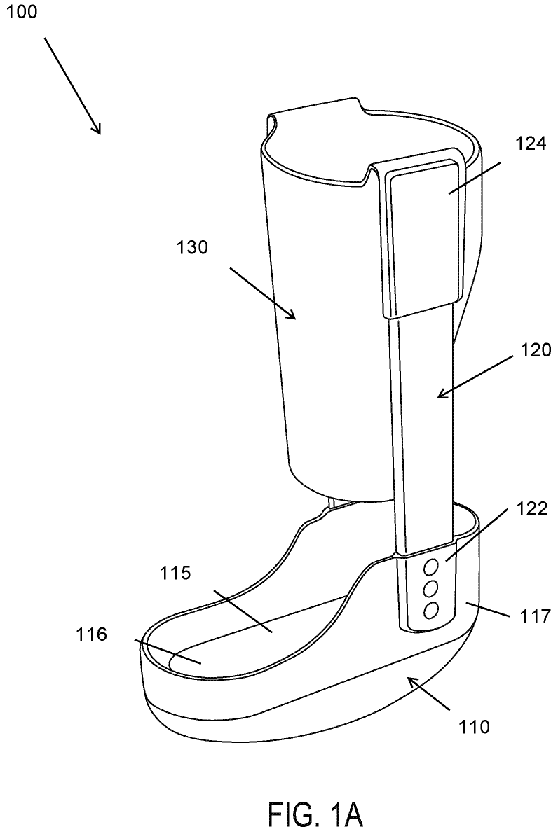

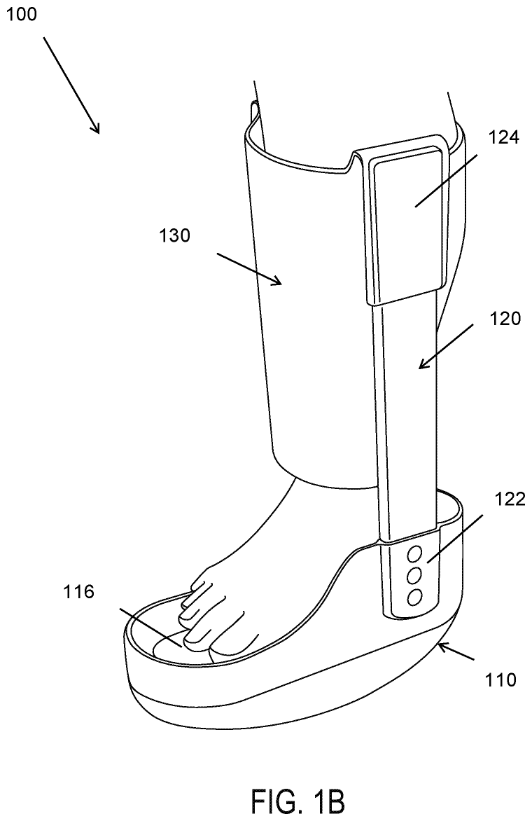

FIG. 1A illustrates an embodiment of a foot suspension system 100 having a vertical bar 120 configured to support a sleeve 130 above the boot 110. The foot suspension system 100 may offload weight from a foot of a user and may include a boot 110, a sleeve 130, and a vertical bar 120 connecting the sleeve 130 and the boot 110. The boot 110 may include a bottom inner surface 115 and may be shaped to receive at least a part of the foot of the user. The boot 110 may include a sleeve 130 configured to secure to a lower appendage of the user to assist with suspending at least the part of the foot above the bottom inner surface 115 of the boot 110. The foot suspension system 100 may include a vertical bar 120 configured to couple the sleeve 130 and the boot 110 together.

The boot 110 may include a bottom inner surface 115 and the boot 110 may be shaped to receive at least a part of the foot of the user. The bottom inner surface 115 may align with the toes, pad, and heel of the user foot. The boot 110 may include a toe area 116 and rear area 117. The toe area 116 may include a volume that allows the user toes to move or pivot freely, including without touching at least the bottom of the toes to the bottom inner surface 115, or the toe area 116 may be sized and shaped to constrain movement of the toes. The rear area 117 of the boot 110 may be positioned to be proximate to the ankle of the user and immobilize the ankle of the user. In some embodiments, the rear area 117 may include one or more features for assisting with ankle rotation and/or extension/flexion of the foot. For example, the rear area 117 may include a spring or other biasing member configured to allow some controlled motion of the ankle.

In some embodiments, the toe area 116 and the rear area 117 of the boot 110 may include a solid piece of material. Alternatively, the toe area 116 and the rear area 117 of the boot 110 may include one or more openings for ventilation.

The bottom inner surface 115 may be sloped. For example, the bottom inner surface 115 115 may be sloped downward from the rear area 117 to the toe area 116. In some embodiments, the toe area 116 may include cushioning to adjust the slope of the bottom inner surface 115. For example, the toe area 116 may include removable cushioning tabs configured to adjust the slope or to provide additional support of the toes and/or midfoot region. In some embodiments, the bottom inner surface 115 may slope upwards from the rear area 117 to the toe area 116. In some embodiments, the rear area 117 may include a different type of cushioning than the toe area 116.

The sleeve 130 may be configured to secure to a lower appendage of the user to assist with suspending the user foot above the bottom inner surface 115 of the boot 110. The sleeve 130 may include a rigid tapered structure that is configured to circumvent a lower appendage (e.g., leg, ankle). The sleeve 130 may secure to the lower appendage (e.g., leg, ankle) through friction and/or compression against the lower appendage (e.g., leg, ankle). The sleeve 130 may have a tapered cylindrical shape or a conical shape. Securing the sleeve 130 to the lower appendage causes the user weight to shift to the foot suspension system 100. The sleeve may secure to the lower appendage by providing one or more of a compression force and a frictional force. In some embodiments, the sleeve 130 may maintain a first distance from the bottom inner surface 115. The first distance is maintained by the vertical bar 120 to shift the user weight from the foot to the foot suspension system 100. The vertical bar 120 maintains the sleeve 130 at the first distance as the user walks in the boot 110. In some embodiments, the first distance may be defined as the distance from the bottom inner surface 115 to the top of the sleeve 130 or the bottom of the sleeve 130.

In some embodiments, the first distance may be determined by a predetermined clearance from the bottom of the user foot to the bottom inner surface 115 of the boot 110. The predetermined clearance may maintain the user foot at a certain height above the bottom inner surface 115 of the boot 110. Additionally, the predetermined clearance may minimize any user weight on the foot of the user. The predetermined clearance may be measured as the distance between the bottom inner surface 115 of the boot 110 and the heel of the foot, the toes of the foot, the pad of the foot, or any combination thereof. In some embodiments, the predetermined clearance may be 1 cm, 2 cm, 3 cm, 4 cm, or 5 cm between the heel, toes, or pad of the foot and the bottom inner surface 115 of the boot 110.

In some embodiments, the first distance may minimize any weight placed on the foot. The foot may maintain some contact with the inner surface, causing the foot to carry some user weight. The first distance may be adjusted to provide a transition from the foot suspension system 100 carrying the user weight to the foot bearing a portion of the user weight. The first distance may be determined by the height at which the vertical bar 120 suspends the sleeve 130 when the foot suspension system 100 is loaded or unloaded with the user weight. The sleeve 130 may be configured to secure a lower appendage of a user above the boot 110 and may be configured to partially cover the lower appendage of the user.

The sleeve 130 may include a lacing feature. The lacing feature may be secured to the sleeve 130 and immobilize the lower appendage of the user (e.g., leg, ankle). The lacing feature may enhance the offloading effect of the foot suspension system 100 by ensuring the sleeve 130 is affixed to the user leg or ankle. The lacing feature may include buckles, ratcheting mechanisms, knob tightening mechanisms, hook & loop, cinching methods, shoelaces, spring, magnets, an elastomeric material, a wafer spring, a honeycomb material insert, an in-mold spring, or any combination thereof. The lacing feature may be present on a front side and/or a backside of the sleeve 130.

The sleeve 130 may comprise a continuous material. The sleeve 130 may comprise a combination of wraps, such as an inner sleeve and an outer sleeve. For example, the inner sleeve may be a compressive sleeve and the outer sleeve may interface with the vertical bar 120. In another example, an inner sleeve may be positioned on a calf area of the user and an outer sleeve may be positioned on the outside of the inner sleeve. The outer sleeve may comprise rigid layers and may be configured to interface with the inner sleeve as well as the vertical bar 120. The inner sleeve may include inner foam layers configured to provide comfort and conformation to the lower appendage (e.g., leg, ankle). These additional foam layers may be reinforced with an outer rigid layer configured to add structural support. In some embodiments, the outer rigid layer may maintain the diameter of the inner sleeve to prevent outward expansion. The outer sleeve may comprise a webbed material or have vertical slits to contour the outer sleeve to the inner sleeve or the user leg or ankle. In some embodiments, the outer sleeve may have one or more openings in the outer rigid layer. In some embodiments, the outer sleeve may include a plurality of material segments that are connected to form a continuous piece of material. For example, the material segments may be horizontal strips and vertical strips.

The vertical bar 120 may extend between the sleeve 130 and the boot 110. The vertical bar 120 may include a first end 122 configured to couple to the boot 110 and a second end 124 configured to couple to the sleeve 130. That is, the sleeve 130 may be supported by the vertical bar 120 at the top end of the foot suspension system 100. The vertical bar 120 may be configured to support the sleeve 130 above the boot 110 thereby assisting with suspending at least the part of the foot above the bottom inner surface 115 of the boot 110. The vertical bar 120 may be a continuous bar or a split bar having a U-type configuration. The vertical bar 120 may include a flared top portion at the top end of the sleeve 130. The flared top portion may be configured to provide additional structural support for the sleeve 130. The vertical bar 120 may be configured to connect to the boot 110 at a connection point that may be configured to rotate or absorb downward force. The connection point may be located proximate to the rear area 117 of the boot 110 and located on the outside surface of the boot 110. The boot 110 may include multiple connection points that correspond to the number of vertical bars. For example, the boot 110 may include two connection points for the vertical bar 120 at opposing sides of the rear area 117 of the boot 110.

The vertical bar 120 may be wider at the second end 124 than the first end 122. The vertical bar 120 may taper as the vertical bar extends from the second end 124 to the first end 122. The vertical bar may have a C-shape or V-shape from a top view of the vertical bar. The vertical bar may have the C-shape or the V-shape from the top end of the vertical bar to the bottom end of the vertical bar. The C-shape or the V-shape may reinforce the strength of the vertical bar in the downward direction and prevent the vertical bar from collapsing. The vertical bar 120 may be configured to extend along a lateral side of the boot 110. In some embodiments, the vertical bar 120 may extend along a front or a backside of the boot 110. The vertical bar 120 may have multiple connection points along the top end of the sleeve 130. In some embodiments, the vertical bar 120 may extend over and above the sleeve 130 to provide additional structural support. The vertical bar 120 may include a tamper-evident or tamper-proof feature that may prevent or provide evidence of tempering by the user. The foot suspension system 100 may include a locking mechanism configured to allow a user or a clinician to remove the foot suspension system 100 in case of emergencies. The locking mechanism may be secured by a key, alpha-numerical combination, a Bluetooth device, an RFID device, and/or the like.

FIG. 1B illustrates an example of a user lower appendage coupled to the foot suspension system 100 shown in FIG. 1A. The foot suspension system 100 may include an adjustable vertical bar 120 configured to adjust a distance between the sleeve 130 and the bottom surface of the boot 110 based on leg length and size. The adjustable vertical bar 120 may enable clinicians to select a particular distance between the user foot and the bottom inner surface 115 of the boot 110 thereby maintaining a predetermined clearance between the bottom inner surface 115 of the boot 110 and the user foot. As the foot recovers, the clinician may prescribe a smaller predetermined clearance between the user foot and the bottom inner surface 115 of the boot 110. In some instances, the clinician may prescribe partial contact between the user foot and the bottom inner surface 115 of the boot 110.

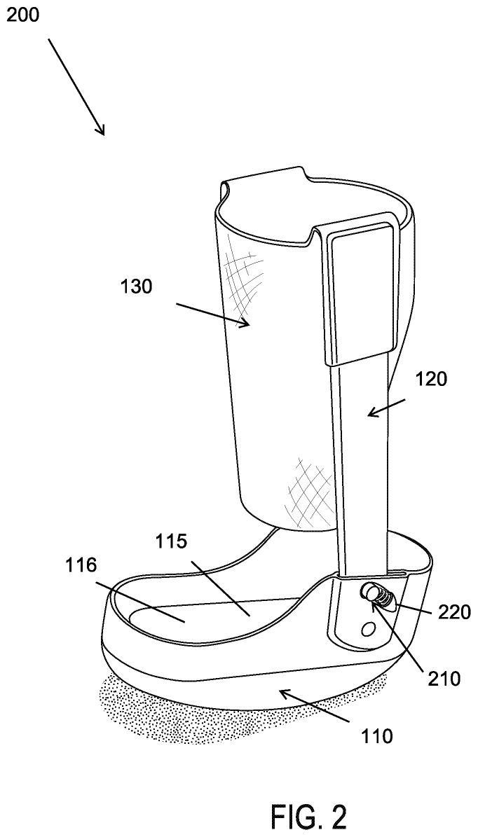

FIG. 2 depicts a foot suspension system 100 having a rotatable pivot 210. The vertical bar 120 may be configured to attach to the rotatable pivot 210 at a connection point between the vertical bar 120 and the boot 110. The rotatable pivot 210 may be configured to allow some rotation of the foot upward and downward. The rotatable pivot 210 may rotatably couple the first end 122 of the vertical bar 120 to the boot 110. The rotatable pivot 210 may allow pivoting of the vertical bar 120 and sleeve 130 relative to the boot 110. The rotatable pivot 210 may enable the sleeve 130 to move with respect to the bottom inner surface 115 of the boot 110 and to enhance a more natural walk.

The rotatable pivot 210 may include a dampening mechanism 220. The dampening mechanism 220 may be configured to dampen the pivoting of the vertical bar 120 and the sleeve 130 relative to the boot 110. The dampening mechanism 220 is at least one of a magnet, an elastomeric material, a spring, a sponge, a wafer spring, a honeycomb insert, and an in-mold spring. The dampening mechanism 220 may be configured to compress when the sleeve 130 is rotated backward relative to the boot 110 and the dampening mechanism 220 may be configured to decompress when the sleeve 130 is rotated forward relative to the boot 110. The dampening mechanism 220 may exert a force on the vertical bar 120 to move the vertical bar 120 to a resting position. For example, when the vertical bar 120 is rotated during a heel strike, the bottom portion of the vertical bar 120 may compress the dampening mechanism 220 to dampen the rotation of the sleeve 130 in a backward direction. When the vertical bar 120 is manipulated in a push off, the dampening mechanism 220 may push back against the bottom portion of the vertical bar 120 to a neutral position.

The desired degree of articulation and flexion/extension resistance may be controlled by using a dampening mechanism 220 of varying density, hardness, or softness. For example, the user may be able to adjust the stiffness of the articulation to their desired level by selecting different dampening mechanisms having different properties, such as density and hardness. In some embodiments, the stiffness of the articulation may be adjusted by limiting the expansion of the dampening mechanism 220 in a first dimension while constraining a second dimension and compressing the material of the dampening mechanism 220 in a third dimension. For example, the method of compressing the material of the dampening mechanism 220 may include but is not limited to using an enclosed space with a wall. Varying the flexion resistance may control the expansion of the dampening mechanism 220 in the first dimension.

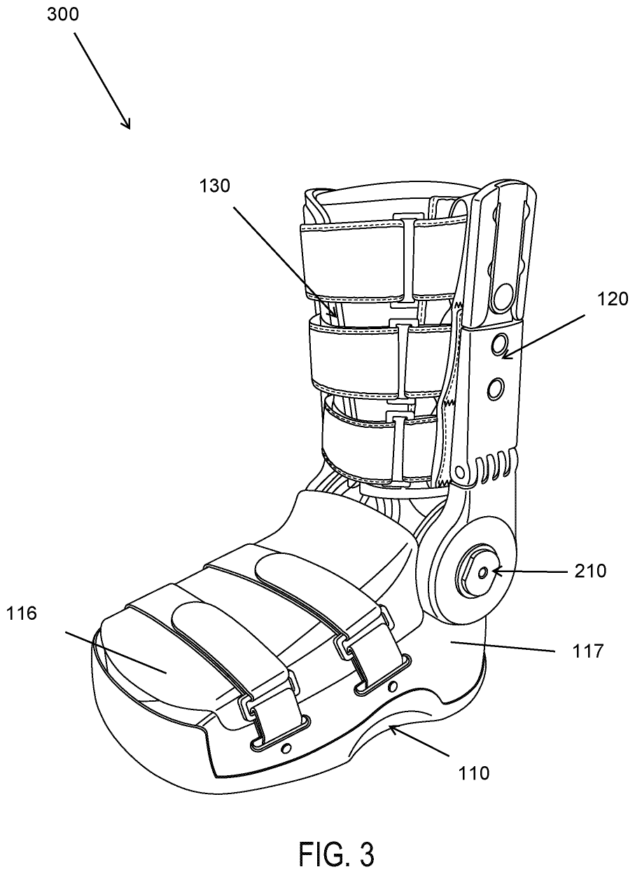

FIG. 3 depicts another foot suspension system 100 having a rotatable pivot 210. The vertical bars may be coupled to a rotatable pivot 210. The rotatable pivot 210 may be located at a rear area 117 of the boot 110. The rotatable pivot 210 may be placed on opposing sides of the boot 110 and additional rotatable pivots may be added to correspond to the number of vertical bars. The rotatable pivot 210 may be configured to couple to the first end 122 of the vertical bar 120. The rotatable pivot 210 may be configured to rotate with the vertical bar 120 as the sleeve 130 moves with respect to the boot 110. For example, the sleeve 130 may pivot backward relative to the boot 110 during the heel strike of a user. In another example, the sleeve 130 may pivot forward relative to the boot 110 during the push off of a user.

The rotatable pivot 210 may be an external attachment to the boot 110 and may pivot on an outside portion of the boot 110. The boot 110 may be shaped to receive a circular portion of the rotatable pivot 210 at the rear area 117 of the boot 110. The rotatable pivot may have the dampening mechanism confined to an area between the boot 110 and the rotatable pivot 210. The dampening mechanism 220 may be configured to dampen the movement of the sleeve 130 with respect to the boot 110. The dampening mechanism 220 may include a torsion spring, a compression spring, and a cushioned insert. In some embodiments, the rotatable pivot 210 may be detachable from the boot 110. The rotatable pivot 210 may have a limited range of motion such that the sleeve 130 does not move beyond a certain angle relative to the boot 110. In some embodiments, the rotatable pivot 210 may be circular and the dampening mechanism 220 contained therein may be pressed against a back portion of the boot 110. The torque resistance may increase as the rotatable pivot 210 presses against the dampening mechanism 220.

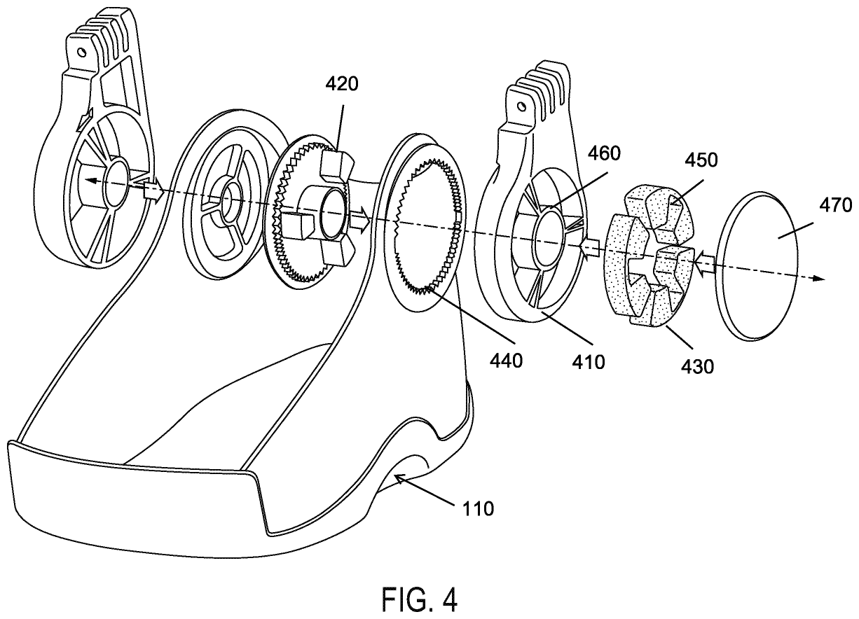

FIG. 4 depicts an exploded view of a rotatable pivot 210. The rotatable pivot 210 may provide an interface between the vertical bar 120 and the boot 110. The rotatable pivot 210 may include an engagement feature 410, a radial protrusion feature 420, and a compressible insert 430. The rotatable pivot 210 may be configured to be inserted into a circular aperture 440 in the boot 110. The engagement feature 410 may be integrated into a lower portion of the vertical bar 120 that is configured to receive the compressible insert 430 and the radial protrusion feature 420. The lower portion of the vertical bar 120 may be flush against an outside portion of the boot 110. The lower portion of the vertical bar 120 may be configured to couple to the circular aperture 440 via the radial protrusion feature 420.

An engagement feature 410 may be integrated into the lower portion of the vertical bar 120 to engage the compressible insert 430. In some embodiments, the engagement feature 410 may be a fin or a spoke connecting to a hub 460 of the lower portion of the vertical bar 120. The engagement feature 410 may extend from the hub 460 to a circumference of the lower portion of the vertical bar 120. The engagement feature 410 may taper as the engagement feature 410 extends from the circumference of the lower portion of the vertical bar 120 to the hub 460. The hub 460 may have an aperture configured to receive the radial protrusion feature 420 from an internal side of the boot 110. Additionally, and/or alternatively, the radial protrusion feature 420 may be inserted into the aperture of the hub 460 from the cap 470. In some embodiments, the engagement feature 410 may not have a hub 460 and the fin may extend from a center of the lower portion of the vertical bar 120.

The engagement feature 410 may be configured to compress the compressible insert 430 as the rotatable pivot 210 rotates with the vertical bar 120 with respect to the boot 110. When the vertical bar 120 is rotated during a heel strike, the engagement feature 410 may compress the compressible insert 430 to dampen the rotation. The engagement feature 410 may compress the compressible insert 430 against the radial protrusion feature 420 or another feature configured to secure the compressible insert 430. When the vertical bar 120 is manipulated in a push off, the compressible insert 430 may push back against the engagement feature 410 to push the engagement feature 410 to a neutral position. In some embodiments, the engagement feature 410 may be rotatable to the boot 110 to compress the compressible insert 430.

The compressible insert 430 may dampen rotational movement and may be situated between one or more engagement features 410. The compressible insert 430 may be formed to receive the engagement feature 410 for allowing the engagement feature 410 to be aligned with the compressible insert 430 in the rotatable pivot 210. The compressible insert 430 may be made from rubber, cork, an elastomeric material, a grommet, and/or the like. The compressible insert 430 may compress when a torque is applied to the vertical bar 120 and the compressible insert 430 may decompress when no torque is applied to the vertical bar 120 to push the engagement feature 410 and rotatable pivot 210 back to a neutral position. The compressible insert 430 may be formed to fit between the hub 460 and one or more engagement features 410. The compressible insert 430 may be configured to articulate forward and backward by squeezing or deforming the compressible insert 430. The compressible insert 430 may decompress to force the vertical bar 120 back to a resting or neutral position.

The desired degree of articulation and flexion resistance may be controlled by using a compressible insert 430 of varying density, hardness, or softness. For example, the stiffness of the articulation may be adjusted to the desired level by selecting different materials for the compressible insert 430 having different properties such as density and hardness. In some embodiments, the stiffness of the articulation may be adjusted by limiting the expansion of the compressible insert 430 in a first dimension while constraining a second dimension and compressing the material of the compressible insert 430 in a third dimension. For example, the method of compressing the material of the compressible insert 430 may include but is not limited to using the cap 470. By thus controlling the expansion of the compressible insert 430 in the first dimension, the flexion resistance may be varied.

The radial protrusion feature 420 may couple the vertical bar 120 to the circular aperture 440 of the boot 110. The radial protrusion feature 420 may be inserted from an inside portion of the boot 110 and include protrusions that are configured to be inserted into the hub 460 and/or between the engagement feature 410 (e.g., spoke, fin). Additionally, and/or alternatively, the radial protrusion feature 420 may be inserted into the aperture of the hub 460 from the cap 470. In some embodiments, the radial protrusion feature 420 may be configured to be inserted into a cutout 450 in the compressible insert 430. For example, the radial protrusion feature 420 may include three protruding features that are each configured to be inserted into the cutout 450 located in a center portion of each compressible insert 430. In another example, the radial protrusion feature 420 may be configured to be inserted into a cutout 450 at a side portion of the compressible insert 430.

The radial protrusion feature 420 may be configured to secure the compressible insert 430 in place at the rotatable pivot 210. The radial protrusion feature 420 may be configured to secure the vertical bar 120 against the boot 110. The radial protrusion feature 420 may include teeth around a circumference to secure the radial protrusion feature 420 to the circular aperture 440. The radial protrusion feature 420 may be fixed by a plurality of teeth in a circular aperture 440 at the rear area 117 of the boot 110 as a torque is applied to the vertical bar 120. In some embodiments, the radial protrusion feature 420 may apply force to the compressible insert 430 as the rotatable pivot 210 rotates with the vertical bar 120 with respect to the boot 110. In some embodiments, the engagement feature 410 may apply a force against the radial protrusion feature 420 as a torque is applied to the vertical bar 120. In some embodiments, the radial protrusion feature 420 has a shape resembling a 3-leaf clover. In some embodiments, the radial protrusion feature 420 may be encapsulated by the vertical bar 120 and the circular aperture 440.

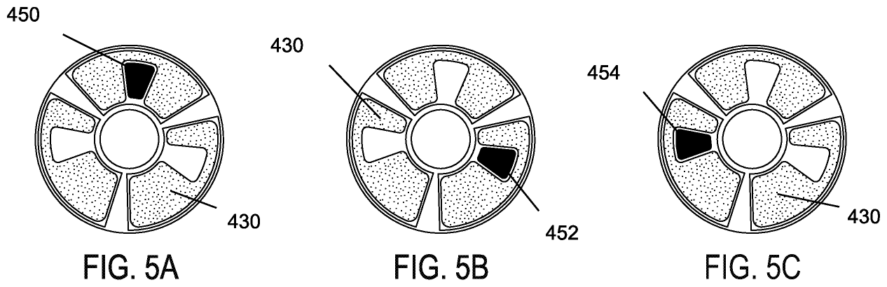

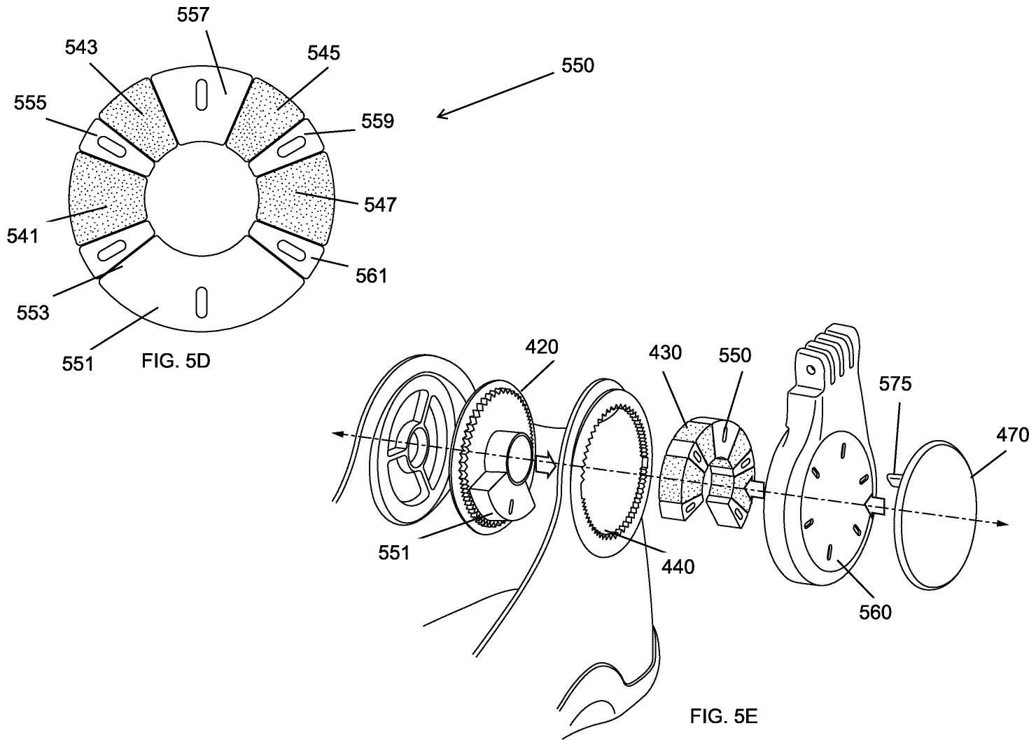

FIG. 5A depicts a configuration of the compressible insert 430 in a rotatable pivot 210. The compressible insert 430 may be situated between one or more engagement features 410. The compressible insert 430 may be formed to receive the engagement feature 410 for allowing the engagement feature 410 to be aligned with the compressible insert 430 in the rotatable pivot 210. The compressible insert 430 may include a cutout 450 to receive the radial protrusion feature 420. As shown in FIG. 5A, the cutout 450 may be in the middle of the compressible insert 430. The cutout 450 in the middle of the compressible insert 430 may enable an equal amount of forward rotation play as backward rotation play. For example, the compressible insert 430 may rotate the vertical bar 120 forward a distance in response to a torque applied in a forward direction. When the cutout 450 in the compressible insert 430 is situated in the center of the compressible insert 430, the compressible insert 430 may rotate the vertical bar 120 backward the same distance in response to the same amount of torque applied in a backward direction. Additionally, when no torque is applied to the compressible insert 430, the compressible insert 430 may push the vertical bar 120 backward to the neutral position at the same time rate as the compressible insert 430 pushes the vertical bar 120 forward to the neutral position. In some embodiments, the cutout 450 may be in the middle of the compressible insert 430 thereby enabling an equal dampening in a forward pivoting direction of the vertical bar 120 and a backward pivoting direction of the vertical bar 120.

FIG. 5B depicts another configuration of the compressible insert 430 in a rotatable pivot 210. The compressible insert 430 may include an offset cutout 452 to receive the radial protrusion feature 420. As shown in FIG. 5B, the offset cutout 452 may be offset from the center of the compressible insert 430. The offset cutout 452 may be offset from a center of the compressible insert 430 for dampening the rotation of the vertical bar 120 in a backward rotation more than a forward rotation of the vertical bar 120. An offset cutout 452 in the compressible insert 430 may enable greater backward rotation play than forward rotation play. For example, the compressible insert 430 may rotate the vertical bar 120 backward a distance in response to a torque applied in a backward direction. When the offset cutout 452 in the compressible insert 430 is offset from the center, the compressible insert 430 may rotate the vertical bar 120 a smaller distance forward than backward in response to the same amount of torque applied in a forward direction. Additionally, when no torque is applied to the compressible insert 430, the compressible insert 430 may push the vertical bar 120 backward (from a forward position) to the neutral position at a slower time rate than the compressible insert 430 pushes the vertical bar 120 forward to the neutral position (from a backward position). In some embodiments, the offset cutout 452 may enable a greater dampening in a backward pivoting direction of the vertical bar 120 than a forward pivoting direction of the vertical bar 120.

In some embodiments, a corresponding radial protrusion feature may be integrated into the cap 470. The cap 470 and the corresponding radial protrusion feature may be selectively removable from the cutout 450 in the compression insert 430 to select another compression insert to adjust the style of dampening. For example, the corresponding radial protrusion feature may be removed from the compression insert of FIG. 5A and be inserted into the offset cutout 452 of compression insert 430 of FIG. 5B. This changes the style of dampening from a balanced dampening to have a greater dampening in a backward pivoting direction of the vertical bar 120 than a forward pivoting direction of the vertical bar 120.

FIG. 5C depicts yet another configuration of the compressible insert 430 in a rotatable pivot 210. The compressible insert 430 may include an offset cutout 454 to receive the radial protrusion feature 420. As shown in FIG. 5C, the offset cutout 454 may be offset from the center of the compressible insert 430. The offset cutout 454 may be offset from a center of the compressible insert 430 for dampening the rotation of the vertical bar 120 in a forward rotation more than a backward rotation of the vertical bar 120. The offset cutout 454 in the compressible insert 430 may enable greater forward rotation play than backward rotation play. For example, the compressible insert 430 may rotate the vertical bar 120 forward a distance in response to a torque applied in a forward direction. The offset cutout 454 may cause the compressible insert 430 may rotate the vertical bar 120 a smaller distance backward than forward in response to the same amount of torque applied in a backward direction. Additionally, when no torque is applied to the compressible insert 430, the compressible insert 430 may push the vertical bar 120 backward (from a forward position) to the neutral position at a slower time rate than the compressible insert 430 pushes the vertical bar 120 forward to the neutral position (from a backward position). In some embodiments, the offset cutout 452 may enable a greater dampening in a forward pivoting direction of the vertical bar 120 than a backward pivoting direction of the vertical bar 120.

In some embodiments, a corresponding radial protrusion feature may be integrated into the cap 470. The cap 470 and the corresponding radial protrusion feature may be selectively removable from the cutout 450 in the compression insert 430 to select another compression insert to adjust the style of dampening. For example, the corresponding radial protrusion feature may be removed from the compression insert of FIG. 5A and be inserted into the offset cutout 454 of compression insert 430 of FIG. 5C. This changes the style of dampening from a balanced dampening to have a greater dampening in a forward pivoting direction of the vertical bar 120 than a backward pivoting direction of the vertical bar 120.

FIG. 5D depicts a configuration of an engagement feature set 550 with multiple compression inserts in a rotatable pivot 210. The engagement feature set 550 may include engagement features that may be selected to customize the rotational dampening based on a user preference. The rotational dampening in the rotatable pivot 210 may range from no rotational dampening to balanced rotational dampening to a biased rotational dampening. For example, the rotatable pivot 210 may include no dampening when a user selects engagement feature 551. In another example, the user may select balanced dampening when the user selects engagement feature 557. In yet another example, the user may select biased rotational dampening with engagement feature 553, engagement feature 555, engagement feature 559, and engagement feature 561. Each engagement feature in the engagement feature set 550 may include an aperture by which the engagement feature is locked into place. The compression inserts may be situated between the engagement features. For example, compression insert 541 may be inserted between engagement feature 553 and engagement feature 555. In another example, the compression insert 547 may be inserted between engagement feature 559 and engagement feature 561.

Engagement feature 551 may be locked into place by inserting a coupling mechanism into its corresponding aperture. Engagement feature 551 may be configured to prevent rotational dampening of the rotatable pivot 210 in either direction. The engagement feature 553 may be unable to rotate in either direction because engagement feature 553 and engagement feature 555 are situated on both sides of engagement feature 551. In some embodiments, engagement feature 551 may be a protrusion of the radial protrusion feature 420. The radial protrusion feature 420 may be fixed. The radial protrusion feature 420 may include teeth around a circumference to secure the radial protrusion feature 420 to the circular aperture 440. The radial protrusion feature 420 may be fixed by a plurality of teeth in a circular aperture 440 at the rear area 117 of the boot 110 as a torque is applied to the vertical bar 120. The fixed state of the radial protrusion feature 420 may cause the engagement feature 551 to be fixed thereby preventing rotational dampening in either direction.

Engagement feature 557 may be locked into place by inserting a coupling mechanism into its corresponding aperture. Engagement feature 557 may be configured to enable balanced rotational dampening of the rotatable pivot 210 in either direction. The engagement feature 553 may enable balanced rotational dampening in both directions because the same amount of compressible inserts are on both sides of engagement feature 557. For example, the compressible insert 543 and compressible insert 541 may have the same dampening effect as compressible insert 545 and compressible insert 547, except for the rotational direction. That is, the engagement feature 557 may rotate the vertical bar 120 forward and backward at the same distance in response to the same amount of torque applied in a forward direction and a backward direction based on balanced compressible inserts.

Engagement feature 553 and engagement feature 561 may be separately locked into place by inserting a coupling mechanism into its corresponding aperture. Engagement feature 553 and engagement feature 561 may be configured to enable rotational dampening in one direction but not the other direction. For example, engagement feature 553 may be configured to dampen the rotational pivot in a backward direction because engagement feature 553 may compress at least compressible insert 541. But engagement feature 553 may prevent rotational dampening in a forward direction because no compression insert is available on the opposing side of engagement feature 553. Similarly, engagement feature 561 may be configured to dampen the rotational pivot in a forward direction because engagement feature 561 may compress at least compressible insert 547. But engagement feature 561 may prevent rotational dampening in a forward direction because no compression insert is available on the opposing side of engagement feature 561 and engagement feature 551 is not configured to rotate.

Engagement feature 555 and engagement feature 559 may be separately locked into place by inserting a coupling mechanism into its corresponding aperture. Engagement feature 555 and engagement feature 559 may be configured to enable greater rotational dampening in one direction compared to the other direction. For example, engagement feature 555 may be configured to dampen the rotational pivot in a backward direction more than a forward direction. Engagement feature 555 may be configured to compress compression insert 543, compression insert 545, and compression insert 547 in a backward direction in response to a backward torque applied to the vertical bar 120. Engagement feature 555 may only compress compression insert 541 in a forward direction in response to a forward torque applied to the vertical bar 120. Similarly, engagement feature 559 may be configured to dampen the rotational pivot in a forward direction more than a backward direction. Engagement feature 559 may be configured to compress compression insert 545, compression insert 543, and compression insert 541 in a forward direction in response to a forward torque applied to the vertical bar 120. Engagement feature 559 may only compress compression insert 547 in a backward direction in response to a backward torque applied to the vertical bar 120.

FIG. 5E depicts another rotatable pivot configured to interface with the embodiment of FIG. 5D. The rotatable pivot 210 may provide an interface between the vertical bar 120 and the boot 110. The rotatable pivot 210 may include an engagement feature set 550, a radial protrusion feature 420, cap 470, and plurality of slots 560. The rotatable pivot 210 may be configured to be inserted into a circular aperture 440 in the boot 110. The engagement feature 410 may be integrated into a lower portion of the vertical bar 120 that is configured to receive the engagement feature set 550 and the radial protrusion feature 420. The lower portion of the vertical bar 120 may be flush against an outside portion of the boot 110. The lower portion of the vertical bar 120 may be configured to couple to the circular aperture 440 via the radial protrusion feature 420.

The engagement feature set 550 may be integrated into the lower portion of the vertical bar 120 to engage the radial protrusion feature 420. The engagement feature set 550 may include engagement features with apertures that may be selected to customize the dampening based on a user preference. The engagement feature with a corresponding aperture may be selected by inserting protrusion 575 of cap 470 through a slot of the plurality of slots 560. Protrusion 575 may be inserted into any of the apertures in the engagement feature set 550. The engagement feature set 550 may be oriented to be inserted into the radial protrusion feature 420. The engagement feature set may be held in the circular aperture 440 by the radial protrusion feature 420 and the vertical bar 120.

The radial protrusion feature 420 may couple the vertical bar 120 to the circular aperture 440 of the boot 110. The radial protrusion feature 420 may be inserted from an inside portion of the boot 110 and include protrusions that are configured to be inserted into the engagement feature set 550. The radial protrusion feature 420 may be fixed. The radial protrusion feature 420 may include teeth around a circumference to secure the radial protrusion feature 420 to the circular aperture 440. The radial protrusion feature 420 may be fixed by a plurality of teeth in a circular aperture 440 at the rear area 117 of the boot 110 as a torque is applied to the vertical bar 120. The fixed state of the radial protrusion feature 420 may cause the engagement feature 551 to be fixed thereby preventing rotational dampening in either direction.

The plurality of slots 560 may be included in a lower portion of the vertical bar 120. The plurality of slots 560 may be configured to align with the apertures found in the engagement feature set 550. The plurality of slots 560 may be ordered in a radial formation along an outside edge of the vertical bar 120. The plurality of slots 560 may be configured to receive protrusions 575 thereby enabling protrusion 575 to pass through a slot of the plurality of slots 560 to secure an engagement feature from the engagement feature set 550. Additionally, the plurality of slots 560 may be configured to rotate with the vertical bar 120 and the selected engagement feature from the engagement feature set 550.

The cap 470 may include the protrusion 575. Protrusion 575 may be attached to an inside edge of cap 470. The cap 470 may be rotatable to insert the protrusion 575 into the desired slot from the plurality of slots 560. The cap 470 may be configured to attach to the vertical bar to secure the protrusion 575 in a slot from the plurality of slots 560.

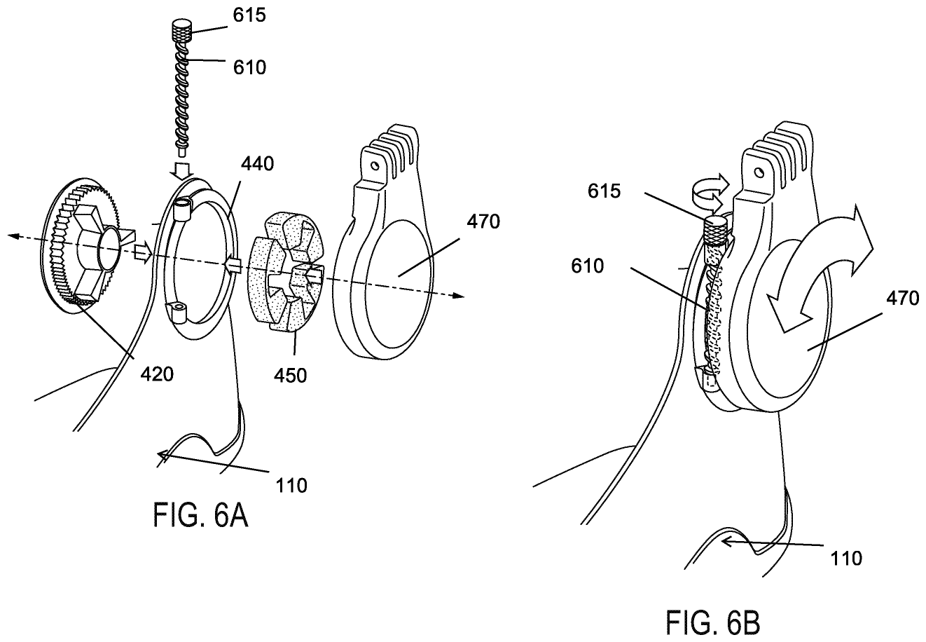

FIG. 6 depicts a gear for adjusting an angle of the vertical bar 120 with respect to the boot 110. The resting angle of the vertical bar 120 with respect to the boot 110 may be adjusted. The resting angle of the vertical bar 120 may enhance user comfort by allowing the foot to pivot at different angles with respect to the user leg. The resting angle may be integrated into the rotatable pivot 210 and may be adjusted by a worm gear 610 rotating the radial protrusion feature 420.

The radial protrusion feature 420 may include a plurality of teeth around a circumference configured to interface with the worm gear 610. The plurality of teeth may be situated around an outer circumference of the radial protrusion feature 420 or an inner circumference of the radial protrusion feature 420. When engaged, the radial protrusion feature 420 may rotate in response to the rotation of the worm gear 610. The worm gear 610 may rotate the radial protrusion feature 420 in a forward direction and a backward direction. Turning the radial protrusion feature 420 may result in rotating the vertical bar 120 thereby adjusting the resting position of the vertical bar 120 with respect to the boot 110. In some embodiments, the radial protrusion feature 420 may be inserted from an inside portion of the boot 110 and include protrusions that are configured to be inserted into the compressible insert 430. The radial protrusion feature 420 may be coupled to an inside portion of the vertical and may secure the vertical bar 120 against the boot 110.

The worm gear 610 may be a linear gear having an exposed end configured to rotate the worm gear 610. The worm gear 610 may be configured to couple to the plurality of teeth and configured to rotate the first gear for adjusting an angle of the vertical bar 120 with respect to the boot 110. The worm gear 610 may be vertically aligned with respect to the boot 110 and may be configured to be inserted between at least two small openings proximate to the circular aperture 440. In some embodiments, the small openings may protrude from the side of the boot 110 and have a diameter larger than the worm gear 610 thereby allowing the worm gear 610 to pass through the small openings.

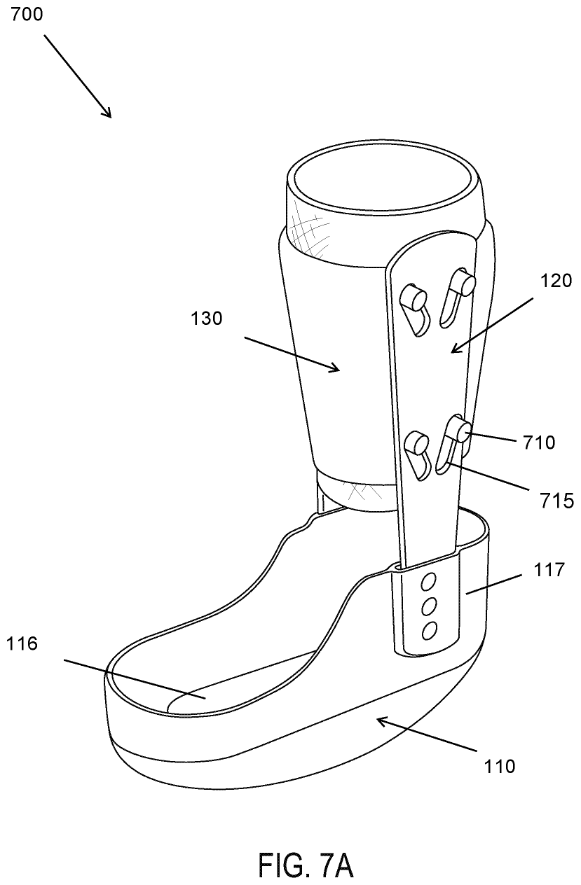

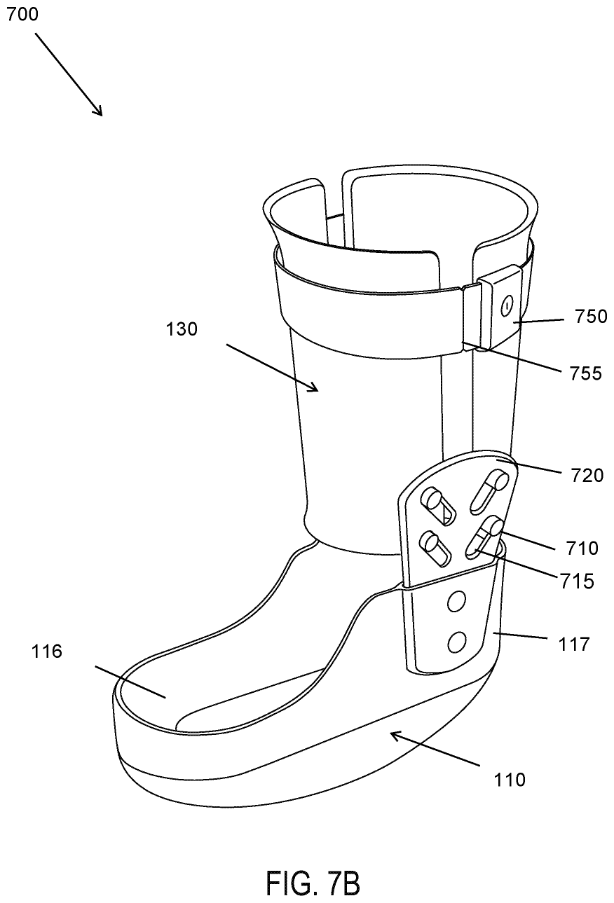

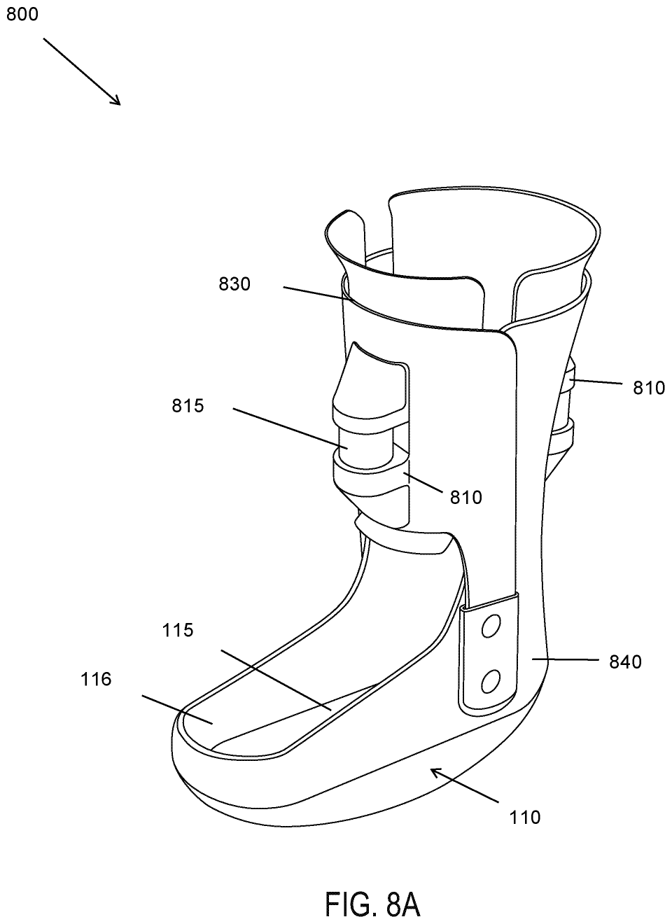

FIG. 7A depicts a foot suspension system 700 having a vertical bar 120 configured to absorb a downward force applied to the sleeve 130. The foot suspension system 100 may include a cushioning mechanism 710 that may reduce the impact force when the heel of the user strikes the ground during the user heel strike. Additionally, the cushioning mechanism 710 may release compression forces when the user lifts the foot suspension system 100 during the push off. The cushioning mechanism 710 may include a spring, a magnet, an elastomeric material, a wafer spring, a honeycomb insert, an in-mold spring, or the like.

The cushioning mechanism 710 may be located at the vertical bar 120. The cushioning mechanism 710 may be configured to move downward in response to a force applied at the sleeve 130 thereby absorbing downward force applied to the sleeve. The cushioning mechanism 710 may absorb the downward force applied to the sleeve such that the downward force of the weight of the user is not transferred to the foot suspension system 100. Additionally, the foot suspension system 100 may include angled vertical opening 715 that may be wide at a first end and may become progressively narrower toward a second end opposite the first end. The angled vertical opening 715 may be an oblong aperture in the vertical bar 120. The sleeve 130 may include a front portion and/or a rear portion that may include the cushioning mechanism 710 and be configured to couple to the angled vertical opening 715 in the vertical bar 120. The cushioning mechanism 710 in the front portion and/or a rear portion of the sleeve 130. The front portion and/or the rear portion of the sleeve 130 may include a detent configured to interface with the cushioning mechanism 710 and that is configured to be received by the angled vertical openings 715 in the vertical bar 120.

The angled vertical opening 715 may be configured to provide absorption space to the cushioning mechanism 710 attached the front and rear portions of the sleeve 130 on the user leg during the user heel strike. The angled vertical opening 715 may be configured to provide space for releasing compression of the front portion and back portion of the sleeve 130 on the user leg during the push off. For example, during the heel strike, the front and back portions of the sleeve 130 may shift downward toward the user foot, and the angled vertical opening 715 may guide the front and rear portions of the sleeve 130 inward to create additional compression forces on the user leg. Additionally, the angled vertical openings 715 may be situated proximate to the rear area 117 of the boot 110 at the more narrow end of the vertical bar 120. The angled vertical opening 715 may allow the cushioning mechanism 710 the travel necessary to absorb the shock without bottoming out at the tapered bottom end of the angled vertical opening 715. In addition to providing a dampening system, the vertical bars may provide a connection between the front and back portions of the sleeve 130 and the boot 110.

FIG. 7B depicts another foot suspension system 700 having a vertical bar 120 configured to absorb a downward force applied to the sleeve 130. In some embodiments, the vertical bar 120 may have a smaller profile to support an ankle sleeve rather than a calf sleeve. For example, the vertical bar 120 may only extend up to an ankle portion of the user foot. In some embodiments, the angled, oblong holes may be situated on the vertical bar 120 having the smaller profile. For example, the angled vertical openings 715 for the cushioning mechanism 710 may be proximate to an ankle portion of the user foot.

The foot suspension system 100 may include a short vertical bar 720 with one end configured to couple to a front portion and/or rear portion of the sleeve 130 and the rear area 117 of the boot 110 at the opposing end. The short vertical bar 720 may include the angled vertical openings 715 that may be an oblong aperture in the vertical bar 120. The sleeve 130 may include a front portion and/or a rear portion that may include the cushioning mechanism 710 and be configured to couple to the angled vertical opening 715 in the vertical bar 120. The cushioning mechanism 710 in the front portion and/or a rear portion of the sleeve 130. The front portion and/or the rear portion of the sleeve 130 may include a detent configured to interface with the cushioning mechanism 710 and that is configured to be received by the angled vertical openings 715 in the vertical bar 120. The angled vertical opening 715 may be angled with a wide first end that tapers toward a narrower second end opposite the first end. The short vertical bar 720 may have a lower profile to provide more support to the ankle region for assisting with offloading user weight.

The foot suspension system 100 may further include a tamper-proof feature 750. For example, the sleeve 130 may further include a locking mechanism configured to allow a user or a clinician to remove the foot suspension system 100 in case of emergencies or prevent a user or patient from removing the foot suspension system 100 without a clinician's consent. The locking mechanism may be a key, an alpha-numerical combination, a Bluetooth device, an RFID device, and/or the like. The tamper-proof feature 750 may be configured to wrap around the sleeve 130 to ensure the user does not remove the boot 110 without clinical supervision. In some embodiments, the foot suspension system 100 may include a breakaway section 755. The breakaway section 755 may include a perforated section, an area that may be cut, a fragile section, or any other breakable connection that would provide evidence of severing. In some embodiments, the breakaway section 755 may also function as a tamper-evident feature, as it provides information as to whether the foot suspension system 100 was removed by the user. In some embodiments, the tamper-evident feature may be a sticker, seal, or other piece of material extending between the front and rear portion of the sleeve 130 that is configured to be fragile or breakable. The tamper-evident feature may provide evidence of removal of the foot suspension system 100 by the user.

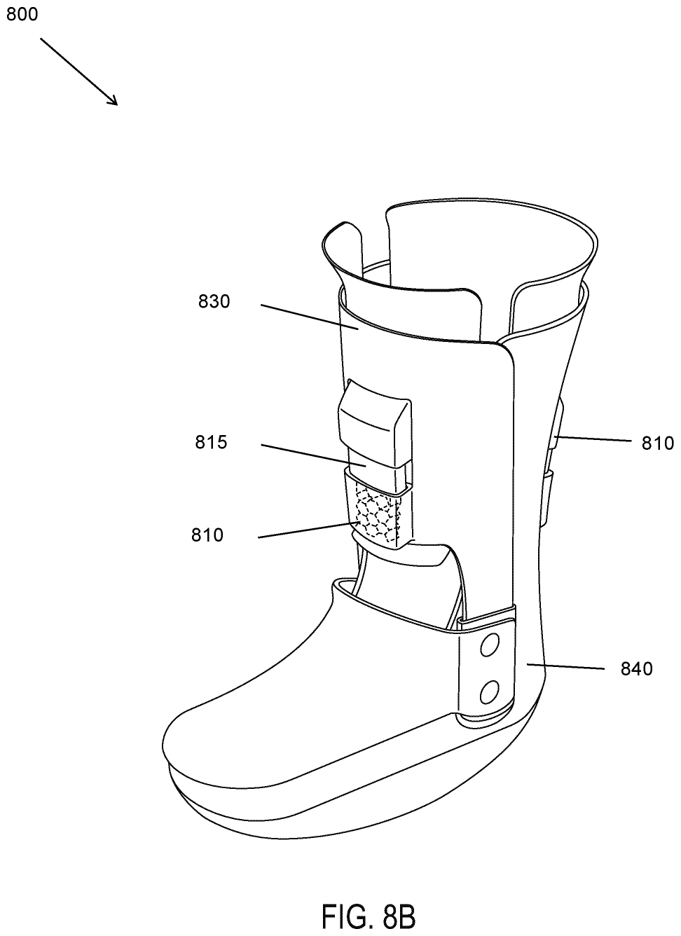

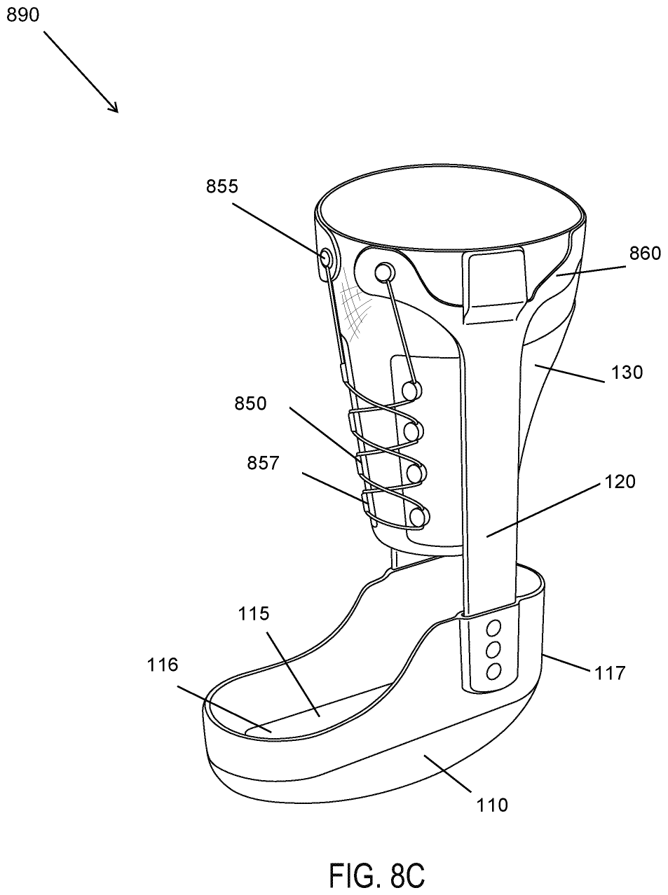

FIG. 8A depicts a foot suspension system 800 having a dampening mechanism 220 inserted into an aperture at a front portion and a back portion of the vertical bar 120. The foot suspension system 100 may include a shock absorber 815 that may reduce the downward force when the heel of the user strikes the ground during the user heel strike. Additionally, the shock absorber 815 may release compression forces when the user lifts the boot 110 during the push off, which releases the weight applied to the boot 110. The shock absorber 815 may include a spring, a magnet, an elastomeric material, a wafer spring, a honeycomb insert, an in-mold spring, and/or the like. The shock absorber 815 may be configured to dampen movement of the vertical bar 120 with respect to the boot 110.

The shock absorber 815 may be an external feature 810 to the vertical bar 120 or the boot 110. The shock absorber 815 may be configured to absorb a downward force from the sleeve 130. The external feature 810 may include a top restraining portion and a bottom restraining portion that forms an aperture. The shock absorber 815 may be inserted into the aperture. The top restraining portion may extend from a top end of the vertical bar 120 and the bottom restraining portion may extend from a bottom end of the vertical bar 120. The top restraining portion and the bottom restraining portion may house the shock absorber 815. The external feature 810 may have an enclosed configuration or a partially exposed configuration (as shown in FIG. 8A). The top restraining portion and the bottom restraining portion may include approximately horizontal surfaces for housing the shock absorber 815 in the partially exposed configuration.

In some embodiments, the shock absorber 815 may be located on a front-facing vertical bar 830 and a raised rear portion 840 of the boot 110. The shock absorber 815 may be configured to absorb downward forces at the front-facing vertical bar 830 and the raised rear portion 840 of the boot 110. The shock absorber 815 at the front-facing vertical bar 830 may absorb a downward force as the user weight shifts forward in the sleeve 130. The shock absorber 815 at the raised rear portion 840 may absorb a downward force during the user heel strike. The shock absorber 815 of the front-facing vertical bar 830 and the raised rear portion 840 may decompress during the user step off.

In some embodiments, the shock absorber 815 of the front-facing vertical bar 830 and the raised rear portion 840 may be configured to absorb a force from the front and back portion of the sleeve 130. For example, when the user is on the heel strike, the front and back portions of the sleeve 130 may shift downward toward the user foot, and the shock absorber 815 may absorb the downward movement of the front and rear portions of the sleeve 130. In some embodiments, the vertical bar 120 may be partially flexible with the shock absorber 815 configured to absorb the flexions of the front-facing vertical bar 830 and the raised rear portion 840. In addition to providing a dampening system, the front-facing vertical bar 830 and the raised rear portion 840 provide an interface between the front and back portions of the sleeve 130 and the boot 110. The front portion of the vertical bar 120 may cover a front side of the sleeve 130 and the back portion of the vertical bar 120 may cover a backside of the sleeve 130.

FIG. 8B depicts a foot suspension system 800 shown in FIG. 8A having another shock absorber 815 inserted into an aperture at a front portion and a back portion of the vertical bar 120.

FIG. 8C depicts a side perspective view of a foot suspension system 890 having a sleeve 130 with a lacing feature 850. The lacing feature 850 may be configured to compress the sleeve during the heel strike and decompress the sleeve during the push off via a pulley-like mechanism 855. The lacing feature 850 may be secured to the sleeve 130 and immobilize the lower appendage of the user (e.g., leg, ankle). The lacing feature 850 may enhance the offloading effect of the foot suspension system 100 by ensuring the sleeve 130 is affixed to the user leg or ankle. The pulley-like mechanism 855 may be attached to an upper portion of the sleeve 130 or vertical bar 120. In some embodiments, the pulley-like mechanism 855 may be connected to a flared portion 860 of the vertical bar 120. In some embodiments, the pulley-like mechanism 855 may automatically tighten and loosen as the user walks. For example, the pulley-like mechanism 855 may add compression to the sleeve 130 on the user heel strike and decompress the sleeve 130 on the user push off. For example, the pulley-like mechanism 855 may automatically tighten during a user heel strike at a first end of the laces 857 and at a second end of the laces 857 that is opposite the first end. The sleeve 130 may be automatically compressed by the lacing feature 850 by allowing the first end of the laces 857 and the second end of the laces 857 to move closer together. The pulley-like mechanism 855 may be configured to provide maximum compression against the user leg or ankle during a heel strike. Additionally, the sleeve 130 may automatically decompress the lacing feature 850 during the user push off by allowing the first end of the laces and the second end of the laces to move further away from each other. The pulley-like mechanism 855 may be configured to provide minimal compression against the user leg or ankle when the user is in a push off.

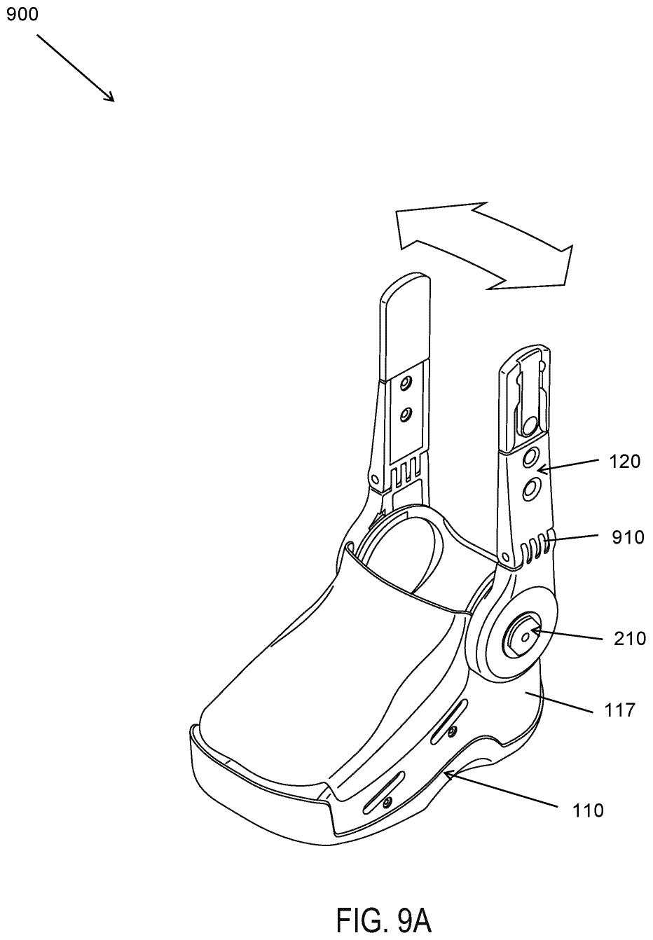

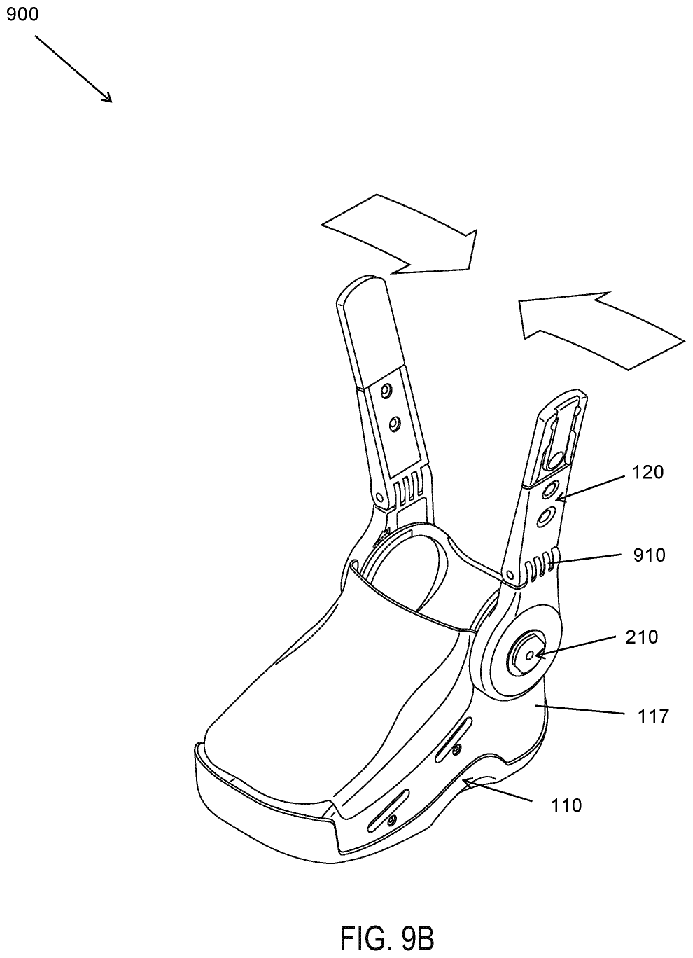

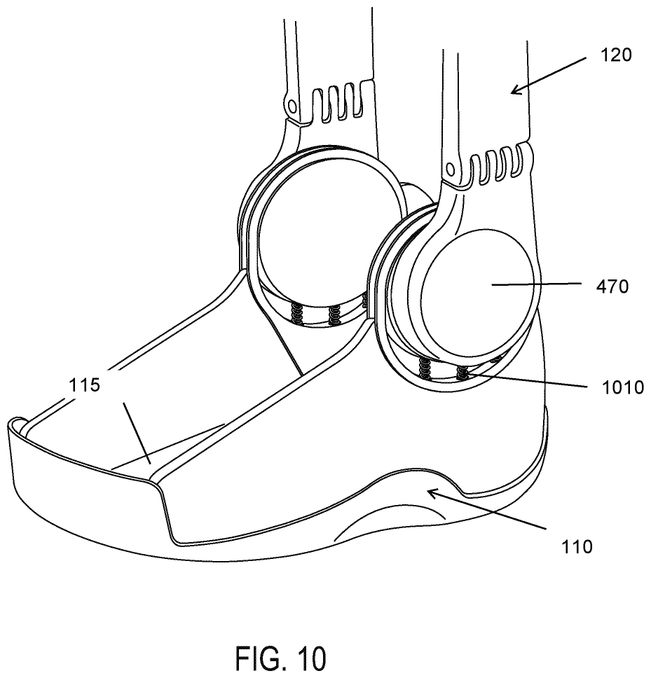

FIG. 9A depicts a foot suspension system 100 having a hinge 910 configured to pivot the vertical bar 120 in a direction away from the boot 110. A hinge 910 may be included on the vertical bar 120. The hinge 910 may be located above the rotatable pivot 210 on the boot 110. The hinge 910 may be configured to rotate the vertical bar 120 in a direction away from the boot 110 in response to force applied at the sleeve 130.

In at least one embodiment, the hinge 910 of the vertical bar 120 may be oriented upwards at a 90-degree angle. The hinge 910 of the vertical bar 120 may be configured to bend outward at an angle when the user leg is inserted into the sleeve 130. The hinge 910 may be fixed into place by tightening a bolt at the hinge. Additionally, and/or alternatively, the hinge 910 may apply a lateral force to return to the upright position when the user leg is not inserted in the sleeve 130. In some embodiments, the hinge 910 may allow the foot suspension system 100 to adapt to different shapes and sizes of lower appendages. For example, the hinge 910 may bend further outwards in response to a wider calf being inserted into the sleeve 130. In some embodiments, the hinge 910 may increase the likelihood that the sleeve 130 fits snugly against the lower appendage of the user. The hinge 910 may have a bolt that is tightened to ensure the sleeve 130 fits snugly against the lower appendage of the user. In some embodiments, the hinge 910 may apply a lateral inward force to increase the likelihood that the sleeve 130 fits snugly against the lower appendage of the user. The hinge 910 may comprise spaced protrusions that are configured to interlace with one another at a connection point.

FIG. 9B depicts an embodiment of FIG. 9A having a hinge 910 rotated away from the boot 110. The hinge 910 may be fixed to the rotatable pivot 210 or vertical bar 120. In some embodiments, the hinge 910 may apply a lateral inward force when bent outward to hold the sleeve 130 in position. In some embodiments, the hinge 910 may apply a lateral inward force to return to the upright position while the hinge 910 is bent outward due to the user leg being inserted in the sleeve 130. The hinge 910 may be configured to extend the vertical bar 120 at an angle away from the boot 110.

FIG. 10 depicts a vertically oriented spring 1010 configured to absorb the downward force applied by the sleeve 130. The foot suspension system 100 may include a rotatable pivot 210. The rotatable pivot 210 may be configured to include a spring or a dampening mechanism below the pivot to reduce the impact force when the heel of the user strikes the ground during the user heel strike. Additionally, the vertically oriented spring 1010 may release compression forces when the user lifts the boot 110 during the push off. The vertically oriented spring 1010 may include a magnet, an elastomeric material, a wafer spring, a honeycomb insert, an in-mold spring, or the like. The vertically oriented spring 1010 may be configured to dampen movement of the vertical bar 120 with respect to the boot 110.

The vertically oriented spring 1010 may be configured to absorb the downward force applied to the sleeve 130 and may be attached to the rotatable pivot 210. The vertically oriented spring 1010 may be situated in the circular aperture 440 of the boot 110. The vertically oriented spring 1010 may be configured to absorb the downward force from the sleeve 130 and the vertically oriented spring 1010 may be configured to decompress during the user step off. The vertically oriented spring 1010 may reduce the impact of the user weight on the boot 110.

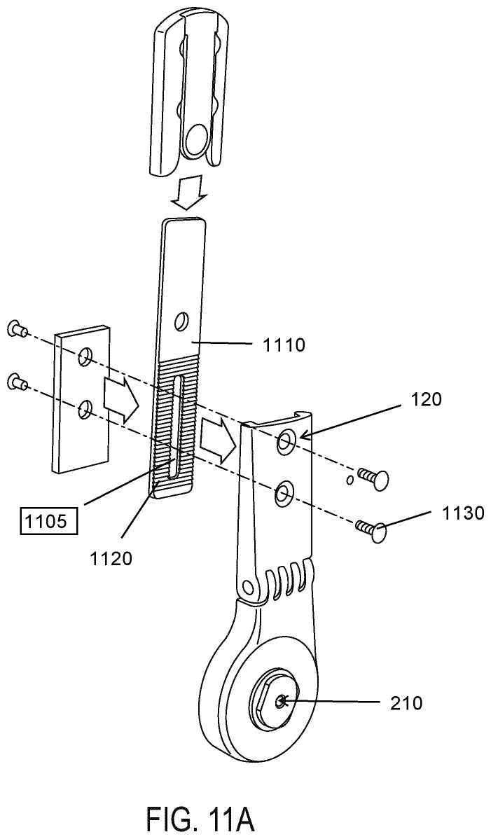

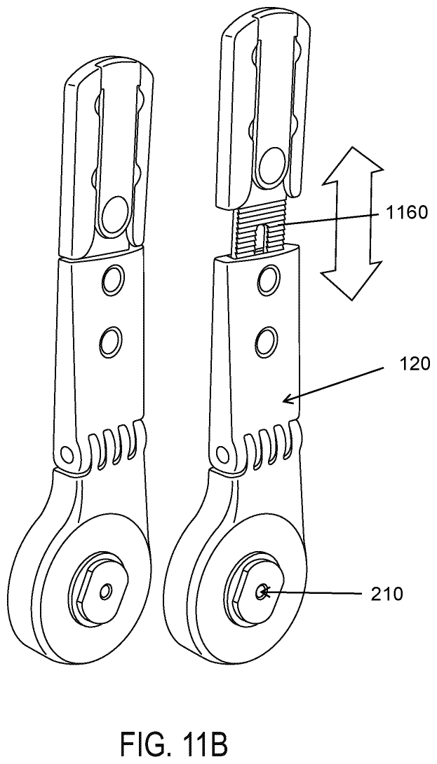

FIG. 11A depicts an adjustable vertical bar 120 having a shaft 1110 and a corrugated surface 1120. The vertical bar 120 may be adjustable to maintain a distance between the sleeve 130 and the bottom inner surface 115 of the boot 110. In some embodiments, the vertical bar 120 may maintain a first distance between the bottom inner surface 115 and the sleeve 130. The vertical bar 120 may maintain the first distance to shift the weight of the user to the foot suspension system 100. The vertical bar 120 may maintain the first distance as the user walks in the boot 110 thereby shifting the user weight to the foot suspension system 100.

The vertical bar 120 may be adjusted using the shaft 1110. The shaft 1110 may be adjusted to increase the likelihood that the user foot maintains a predetermined clearance above the bottom inner surface 115 of the boot 110. The shaft 1110 may maintain the user foot at a predetermined clearance above the bottom inner surface 115 of the boot 110 by securing the shaft 1110 at different heights along the vertical bar 120. The shaft 1110 may be adjusted to a height that allows the foot suspension system 100 to be customized for different users depending on leg length and/or calf size.

The shaft 1110 may be secured at a certain height along the vertical bar 120 using a fastener 1130. In some embodiments, the shaft 1110 may include an elongated aperture 1105 in a mid-section of the shaft 1110 through which a fastener 1130 may pass. The fastener 1130 may pass through an aperture at one side of the vertical bar 120 and pass through the elongated aperture 1105 of the shaft 1110, and then be secured to an opposing side of the vertical bar 120. The fastener 1130 may be a screw, a bolt, a link, a pin, a nail, and/or the like. In some embodiments, the shaft 1110 includes an elongated aperture 1105 in the middle through which a screw is inserted for securing the shaft 1110 to the vertical bar 120. The shaft 1110 may include a corrugated surface 1120 to maintain the shaft 1110 secure against the vertical bar 120. The corrugated surface 1120 may interface with another corrugated surface or a ribbed surface at the vertical bar 120. The corrugated surface 1120 may include horizontal recesses that may be configured to receive horizontal protrusions at the vertical bar 120. Additionally, and/or alternatively, the corrugated surface 1120 may include horizontal protrusions that may be configured to receive horizontal recesses at the vertical bar 120. The corrugated surface 1120 may include teeth that are configured to interface with teeth at the vertical bar 120. The corrugated surface 1120 may include horizontal teeth that may be configured to receive horizontal teeth at the vertical bar 120.

FIG. 11B depicts an assembled view of an adjustable vertical bar 120 of FIG. 11A having a shaft 1110 and a corrugated surface 1120. The shaft 1110 may be inserted into the vertical bar 120. The shaft 1110 may be configured to adjust a height of the vertical bar 120 via a stepping mechanism 1160 (e.g., the corrugated surface 1120). The stepping mechanism 1160 may include a ratchet, a loop, a recess, a protrusion, and/or the like. The shaft 1110 may be adjusted to discrete heights or steps of the stepping mechanism 1160. The shaft 1110 may include a corrugated surface 1120 for immobilizing the shaft 1110 against the vertical bar 120.

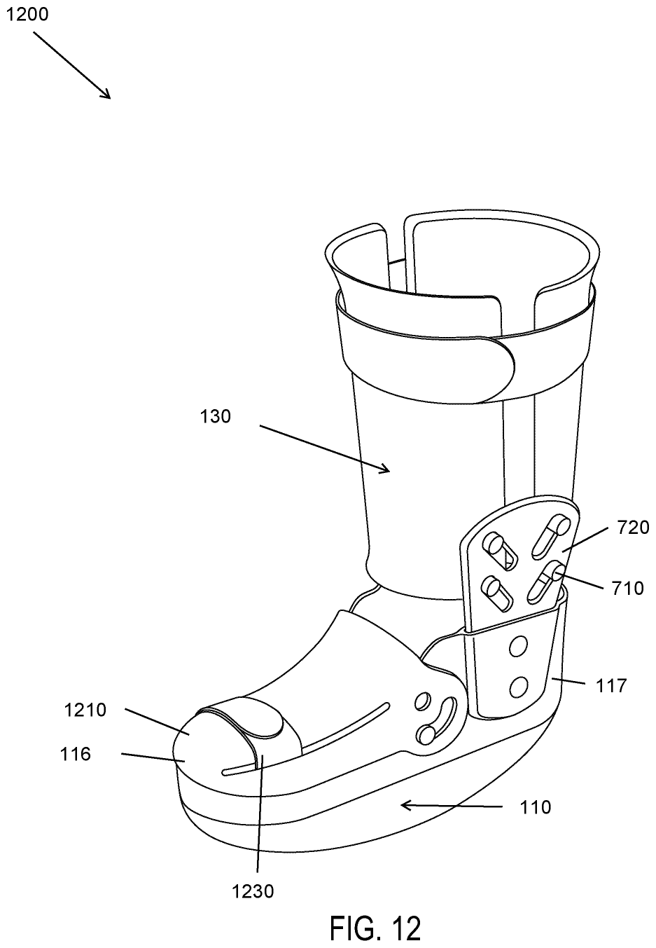

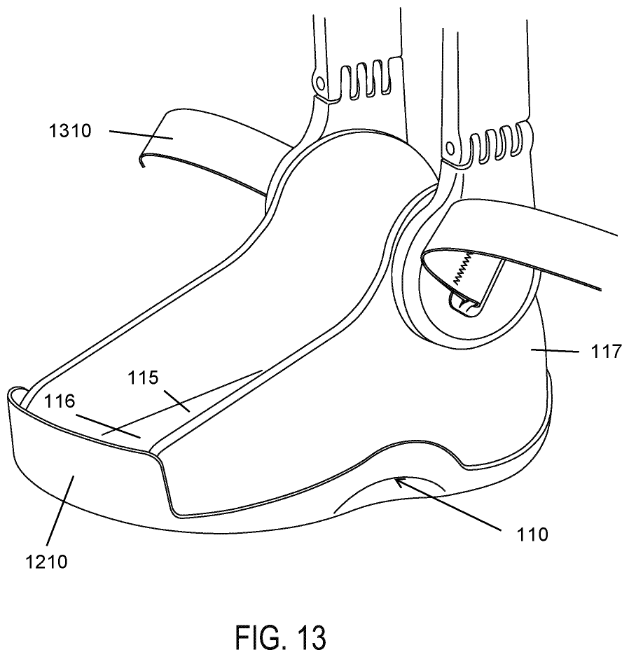

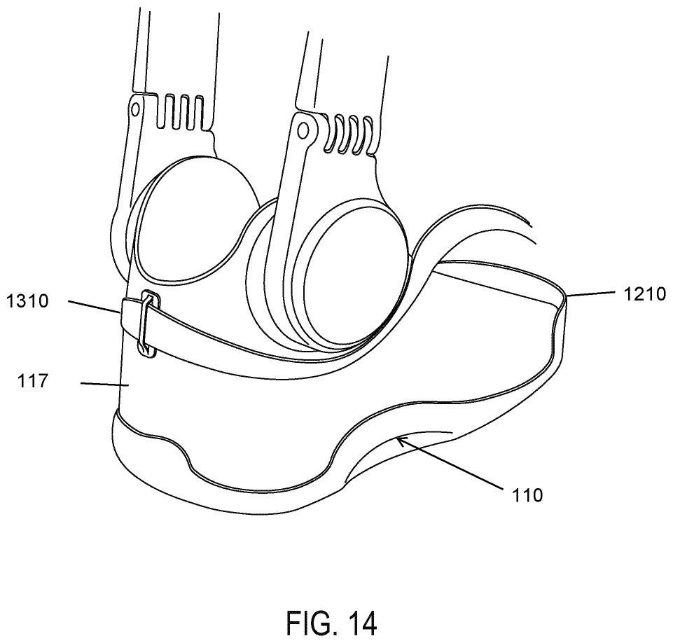

FIG. 12 depicts a dorsal foot covering 1210 configured to pivot to access the toe area 116 of the boot 110. The boot 110 may further include a dorsal foot covering 1210 configured to attach to the boot 110 and cover the foot. Additionally, the dorsal foot covering 1210 may be configured to cover a toe area 116 of the boot 110 and attach to a rear area 117 of the boot 110. The dorsal foot covering 1210 may be configured to pivot to access the toe area 116 of the boot 110. The dorsal foot covering 1210 may be configured to selectively attach to a front lip of the toe area 116. The dorsal foot covering 1210 may include an opening through which an anchoring feature 1230 may secure the dorsal foot covering 1210 to the boot 110.

In some embodiments, the dorsal foot covering 1210 may be configured to couple with the boot 110 at a pivoting connection point configured to allow the dorsal foot covering 1210 to be moved between a raised and a lowered position. In some embodiments, the dorsal foot covering 1210 may include an anchoring feature 1230 configured to hold the user foot at a 90-degree angle relative to the user leg. The anchoring feature 1230 may utilize the pivoting connection point to fix the angle of the boot 110. The anchoring feature 1230 may reduce fatigue or strain on the foot and/or ankle. The anchoring feature 1230 may be an insert in the boot 110. The anchoring feature 1230 may be configured to pass through an opening in the dorsal foot covering 1210. The anchoring feature 1230 may pass through the opening and be extend across an outside surface of the dorsal foot covering 1210. The anchoring feature 1230 may include a strap, basket, net, rigid support, and/or the like to support the forefoot of the user. In some embodiments, the anchoring feature 1230 may couple to the pivoting hinge 910 of the boot 110. The anchoring feature 1230 may be integrated into the dorsal foot covering 1210 as a continuous piece or the anchoring feature 1230 may be releasably coupled to the dorsal foot covering 1210.

In some embodiments, the boot 110 and the dorsal foot covering 1210 may be a continuous piece of material. The rigid foot cover may be configured to extend past an ankle region of the user to approximately a mid-shin area of the user leg. The suspension system may include a removable back cover and a rear outer sleeve. The rear outer sleeve may be configured to couple to the dorsal foot covering 1210 and/or the removable back cover. The removable back cover may be configured to attach to the boot 110 and to the rear outer sleeve.

FIG. 13 depicts a boot 110 having a strapping feature 1310 configured to wrap around the dorsal foot covering 1210 and coupled to a rotatable pivot 210. The boot 110 may include a strapping feature 1310 to secure the user ankle and/or the dorsal foot covering 1210 to the boot 110. The strapping feature 1310 may be disassembled to access the dorsal foot covering 1210. The dorsal foot covering 1210 may then be pivoted to access the user foot. The strapping feature 1310 may emerge from the pivoting hinge 910. The strapping feature 1310 may wrap around the backside of the boot 110. The strapping feature 1310 may be releasably attached to the dorsal foot covering 1210. Additionally and/or alternatively, the strapping feature 1310 may be attached to the ankle or calf region of the user and may extend to the forefoot.

FIG. 14 depicts a boot 110 having a strapping feature 1310 configured to wrap around the dorsal foot covering 1210 and configured to attach to a backside of the boot 110. The strapping feature 1310 may couple to the backside of the boot 110. The strapping feature 1310 may be integrated into the dorsal foot covering 1210 as a continuous piece or the strapping feature 1310 may be releasably coupled to the dorsal foot covering 1210. The strapping feature 1310 may wrap around the backside of the boot 110. The strapping feature 1310 may be releasably attached to the dorsal foot covering 1210. Additionally and/or alternatively, the strapping feature 1310 may be attached to the ankle or calf region of the user and may extend to the dorsal foot covering 1210.

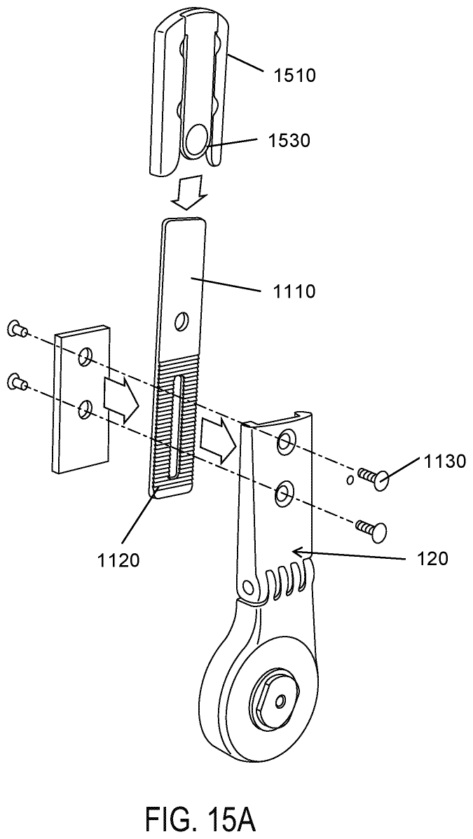

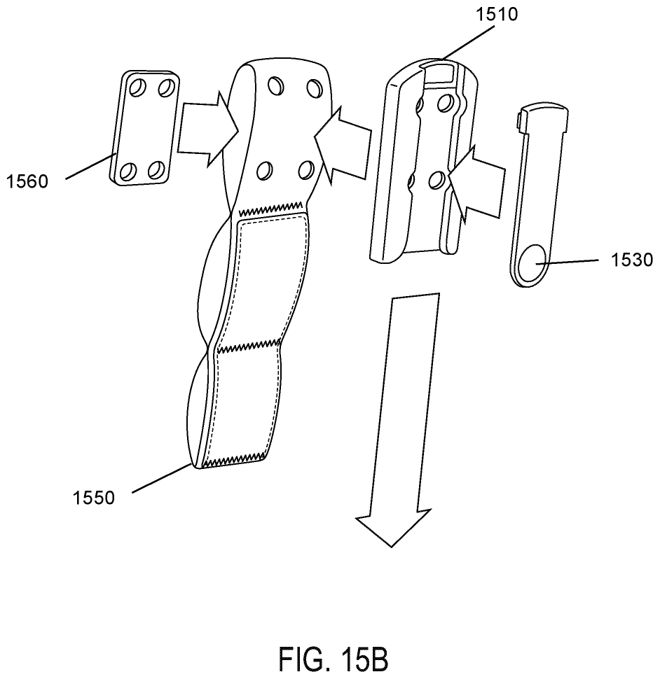

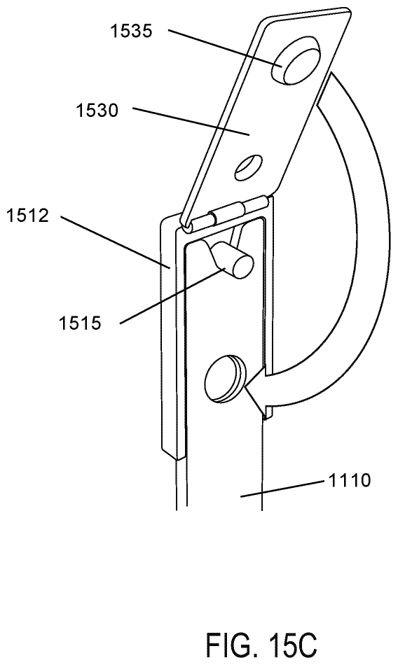

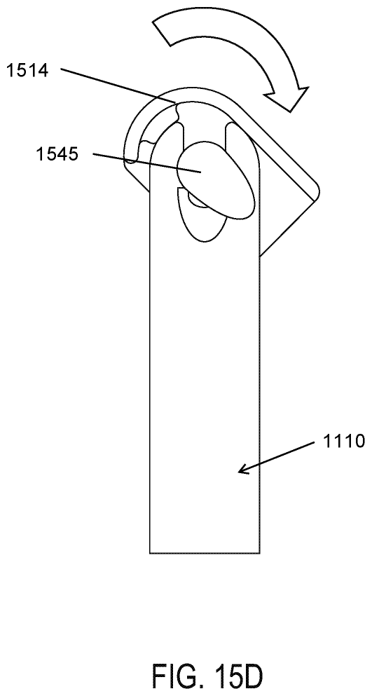

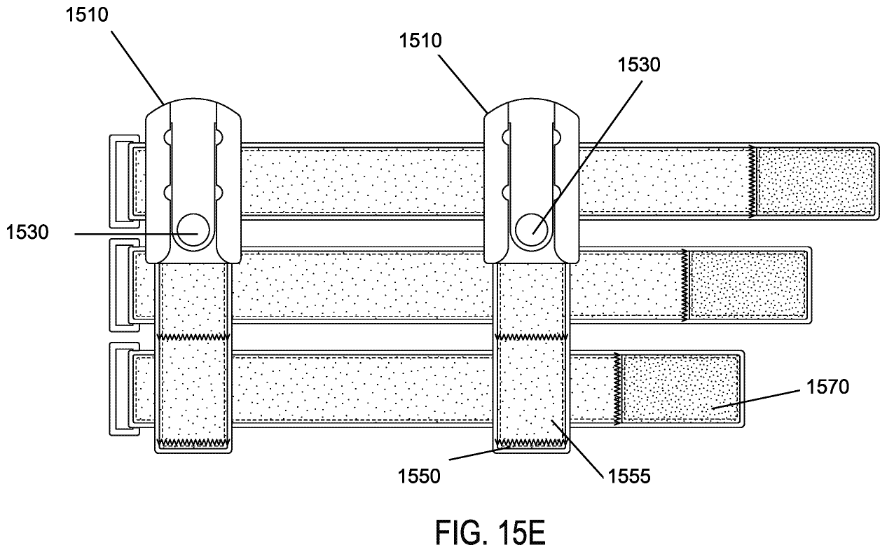

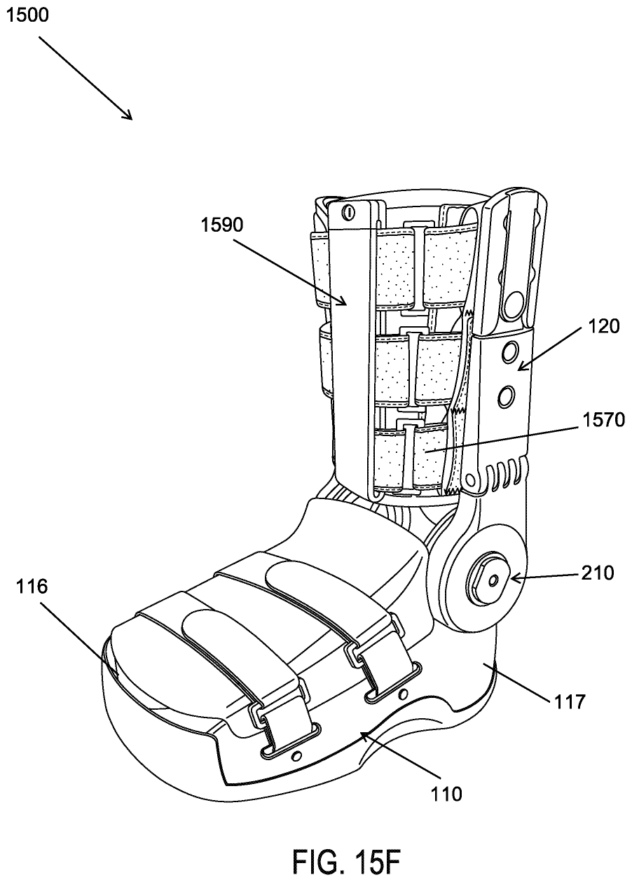

FIG. 15A depicts a foot suspension system 100 having a sleeve anchor 1510 with an aperture for receiving the vertical bar 120. The sleeve anchor 1510 may interface between the sleeve 130 and the vertical bar 120. The sleeve anchor 1510 may be the coupling between the second end of the vertical bar 120 and the sleeve 130. The sleeve anchor 1510 may be situated at a top end of the vertical bar 120 and interface with a top portion of the sleeve 130. The sleeve anchor 1510 may include an opening for receiving the vertical bar 120 and a plurality of openings for fastening the sleeve anchor 1510 to the sleeve 130.

The sleeve anchor 1510 may include an opening for receiving the vertical bar 120. In some embodiments, the opening may receive a shaft 1110 extending from the vertical bar 120. The aperture may be at a bottom end of the sleeve anchor 1510 and a wall may be at the top end of the shaft 1110. The wall may prevent the shaft 1110 and/or vertical bar 120 from sliding through the sleeve anchor 1510. The sleeve anchor 1510 may be a housing configured to encapsulate the shaft 1110 or vertical bar 120. The sleeve anchor 1510 may be configured to selectively detach from the vertical bar 120 for decoupling the sleeve 130 from the vertical bar 120.

The sleeve anchor 1510 may include a protrusion 1520 configured to be inserted into an opening at the vertical bar 120 for securing the sleeve anchor 1510 to the vertical bar 120. The protrusion 1520 may be located at a bottom end of the sleeve anchor 1510. The protrusion 1520 may form part of a covering that is releasably attachable to the sleeve anchor 1510. The protrusion 1520 may be configured to be inserted into multiple openings in the shaft 1110 to adjust the height of the vertical bar 120. The protrusion 1520 may be a knob, a pin, a screw, a bolt, and/or the like.

FIG. 15B depicts an exploded view of a foot suspension system 100 having a sleeve anchor 1510 configured to couple with a sleeve harness 1550. The sleeve anchor 1510 may interface between the sleeve 130 and the vertical bar 120. The sleeve anchor 1510 may be the coupling between the second end of the vertical bar 120 and the sleeve 130. The sleeve anchor 1510 may be situated at a top end of the vertical bar 120 and interface with a top portion of the sleeve 130. The sleeve anchor 1510 may include an opening for receiving the vertical bar 120 and a plurality of openings for fastening the sleeve anchor 1510 to the sleeve 130.

A backside of the sleeve anchor 1510 may include a plurality of openings for interfacing with the sleeve 130. The plurality of openings may be exposed when the releasably attachable covering is disconnected from the sleeve anchor 1510. The plurality of openings may be aligned with a plurality of sleeve 130 openings. A fastener may couple the sleeve anchor 1510 to the sleeve 130, such as a screw, a bolt, a link, a pin, a nail, and/or the like. The fastener may hold the sleeve 130 against the sleeve anchor 1510 by connecting to a plate at an opposite side of the sleeve 130. In some embodiments, the plurality of openings may align with an opening at the sleeve harness 1550 and openings at backplate 1560 thereby allowing the fastener to pass through an opening at the sleeve anchor 1510, the opening at the sleeve harness 1550 to be secured to the opening at the backplate 1560 at the opposite side of the sleeve harness 1550.

The sleeve harness 1550 may include at least one loop for attaching the vertical bar 120 to the sleeve 130. In some embodiments, the sleeve harness 1550 includes at least two loops configured to receive two horizontal straps wrapped around the sleeve 130. The horizontal strap may be configured to be inserted into the loops of the sleeve harness 1550. The sleeve harness 1550 may include a stitching between each of the loops and a surface of the harness may be configured to attach to the sleeve 130. For example, the surface of the harness may include a hook-and-loop interface to attach to the sleeve 130. The sleeve harness 1550 may be configured to connect to the rear area 117 of the boot 110. For example, the sleeve harness 1550 may be coupled to the rear area 117 at a point near the calf of the user, as well as a point near the ankle of the user.

The sleeve harness 1550 may comprise rigid layers and may be configured to interface with the sleeve 130 as well as the vertical bar 120. In some embodiments, the sleeve harness 1550 may be an outer rigid layer configured to maintain a diameter of the sleeve 130 to prevent outward expansion. The sleeve harness 1550 may comprise a webbed material or have vertical slits to contour the sleeve harness 1550 to the sleeve 130 or the user leg or ankle. In some embodiments, the sleeve harness 1550 may have one or more openings. In some embodiments, the sleeve harness 1550 may include a plurality of pieces of materials that are connected to form a continuous piece of material. For example, the pieces of materials may be horizontal strips and vertical strips. The horizontal strips may be hook-and-loop strips that may be configured to attach or pass through the vertical strip.

FIG. 15C depicts a side view of another embodiment of the foot suspension system having another sleeve anchor 1512 for receiving the vertical bar 120. The sleeve anchor 1512 may interface between the sleeve 130 and the vertical bar 120. The sleeve anchor 1512 may couple the second end of the vertical bar 120 to the sleeve 130. The sleeve anchor 1512 may be situated at a top end of the vertical bar 120 and interface with a top portion of the sleeve 130. The sleeve anchor 1512 may include an opening for receiving the vertical bar 120 and the rotating plate 1530 for fastening the vertical bar 120 to the sleeve 130.

The sleeve anchor 1512 may include an opening with a notch 1515 for receiving the vertical bar 120 and/or shaft 1110. The sleeve anchor may also include a rotating plate 1530 with a protrusion 1535 at the end of the rotating plate 1530. In some embodiments, the notch 1515 may be formed to receive an end of the vertical bar 120 to ensure the vertical bar 120 is fixed in the sleeve anchor 1512. The notch 1515 may prevent the shaft 1110 and/or vertical bar 120 from sliding through the sleeve anchor 1510. The sleeve anchor 1512 may be a housing configured to encapsulate the shaft 1110 or vertical bar 120. The rotating plate 1530 may be configured to rotate to encapsulate the shaft 1110 and secure the shaft with the protrusion 1535. The protrusion 1535 may be configured to selectively detach from the vertical bar 120 for decoupling the sleeve 130 from the vertical bar 120.

The rotating plate 1530 may include a protrusion 1535 configured to be inserted into an opening at the vertical bar 120 for securing the sleeve anchor 1512 to the vertical bar 120. The protrusion 1535 may be located at a bottom end of the rotating plate 1530. The protrusion 1535 may be configured to be inserted into multiple openings in the shaft 1110 to adjust the height of the vertical bar 120. The protrusion 1535 may be a knob, a pin, a screw, a bolt, and/or the like.

FIG. 15D depicts another embodiment of the foot suspension system 100 having another sleeve anchor 1514 for receiving the vertical bar 120. The sleeve anchor 1514 may interface between the sleeve 130 and the vertical bar 120. The sleeve anchor 1514 may be the coupling between the second end of the vertical bar 120 and the sleeve 130. The sleeve anchor 1514 may be situated at a top end of the vertical bar 120 and interface with a top portion of the sleeve 130. The sleeve anchor 1514 may include a rotating notch 1545 for receiving the vertical bar 120 for fastening the vertical bar 120 to the sleeve 130.

The sleeve anchor 1514 may include a rotating notch 1545 for receiving the vertical bar 120. The sleeve anchor 1514 may insert the rotating notch into an opening at the vertical bar 120 and/or the shaft 1110 at an angle. Once the sleeve anchor 1514 is inserted into the opening at the vertical bar 120, the rotating notch 1545 may be rotated to secure the sleeve anchor 1514 to the vertical bar 120. Additionally, and/or alternatively, the sleeve anchor 1514 may be rotated to secure the sleeve anchor 1514 to the vertical bar 120 once the sleeve anchor 1514 is inserted into the opening at the vertical bar 120.

FIG. 15E depicts a sleeve anchor assembled to the sleeve harness with the horizontal securement straps. The sleeve harness 1550 may include at least one loop 1555 extending in a vertical direction for attaching the vertical bar 120 to the sleeve 130. In some embodiments, the sleeve harness 1550 including at least one loop 1555 may be configured to receive a horizontal strap 1570 wrapped around the sleeve 130. The horizontal strap 1570 may be configured to be inserted into the at least one loop 1555 of the sleeve harness 1550. The sleeve harness 1550 may include a stitching between each of the at least one loop 1555 and a back surface of the sleeve harness 1550 may be configured to attach to the sleeve 130. For example, the back surface of the sleeve harness 1550 may include a hook-and-loop interface to attach to the sleeve 130. The sleeve harness 1550 may be configured to support the user calf and/or inner sleeve worn by the user. Additionally, and/or alternatively, the sleeve harness 1550 may connect to the rear area 117 of the boot 110. For example, the sleeve harness 1550 may be coupled to the rear area 117 at a point near the calf of the user, as well as a point near the ankle of the user.

FIG. 15F depicts a side view of a foot suspension system 1500 with a locking mechanism 1590 assembled to the sleeve harness 1550. The locking mechanism 1590 may extend over the horizontal strap 1570 to prevent a user or third party from tampering with the foot suspension system 1500. The locking mechanism 1590 may extend parallel to the sleeve harness and include a lock at the top end of the locking mechanism 1590. In some embodiments, the locking mechanism may cinch the horizontal strap 1570 thereby securing the horizontal strap 1570 in place and preventing the horizontal strap 1570 from moving. The locking mechanism 1590 may be a key, an alpha-numerical combination, a Bluetooth device, an RFID device, and/or the like. The locking mechanism 1590 may be configured to prevent a user or patient from removing the foot suspension system 100 without a clinician's consent.

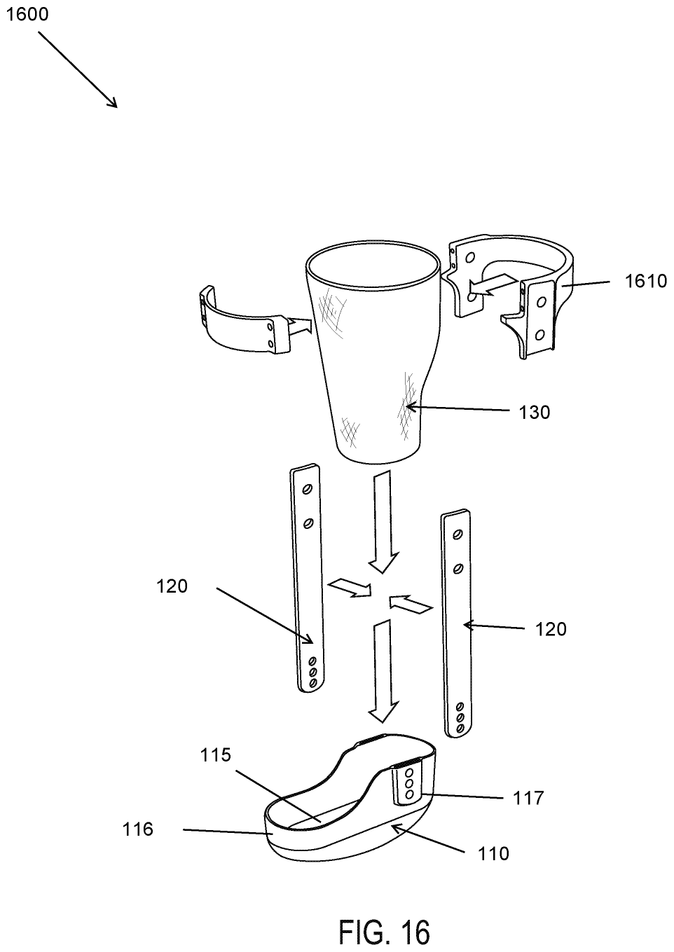

FIG. 16 depicts an exploded view of a foot suspension system 100 having a halo 1610 configured to extend around a circumference of the sleeve 130. The halo 1610 may be configured to circumvent the sleeve 130 to provide additional support for securing the sleeve 130 mechanism to the user ankle or user leg. The halo may couple the vertical bar 120 to the sleeve 130. The halo 1610 may include two side portions at which the vertical bar 120 may be received. The halo 1610 may be coupled to an upper end of the vertical bar 120 and secured to the user leg or ankle with the vertical bar 120.

The halo 1610 may be detachable from the upper portion of the sleeve 130 and include a tightening knob to adjust the compression of the halo 1610 against the sleeve 130. The halo 1610 may have a hollow interior and have a ring formation around the circumference of the sleeve 130. The halo 1610 may be configured to distribute weight of the sleeve uniformly around the halo 1610. The halo 1610 may prevent structural weaknesses in the sleeve 130 where the sleeve 130 couples to the vertical bar 120.

The halo 1610 may be a continuous rigid material or multiple rigid materials. For example, the halo 1610 may include two pieces of rigid material configured to couple to each other as well as the sleeve 130 and the vertical bar 120. The halo 1610 may be configured to provide structural support to maintain the first distance thereby ensuring the foot suspension system 100 is offloading the user weight.

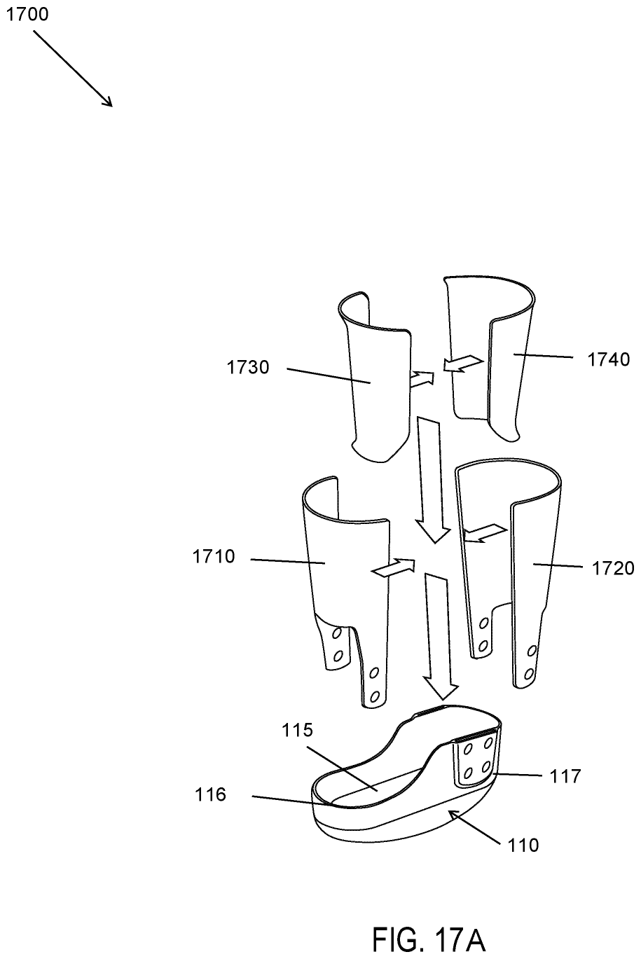

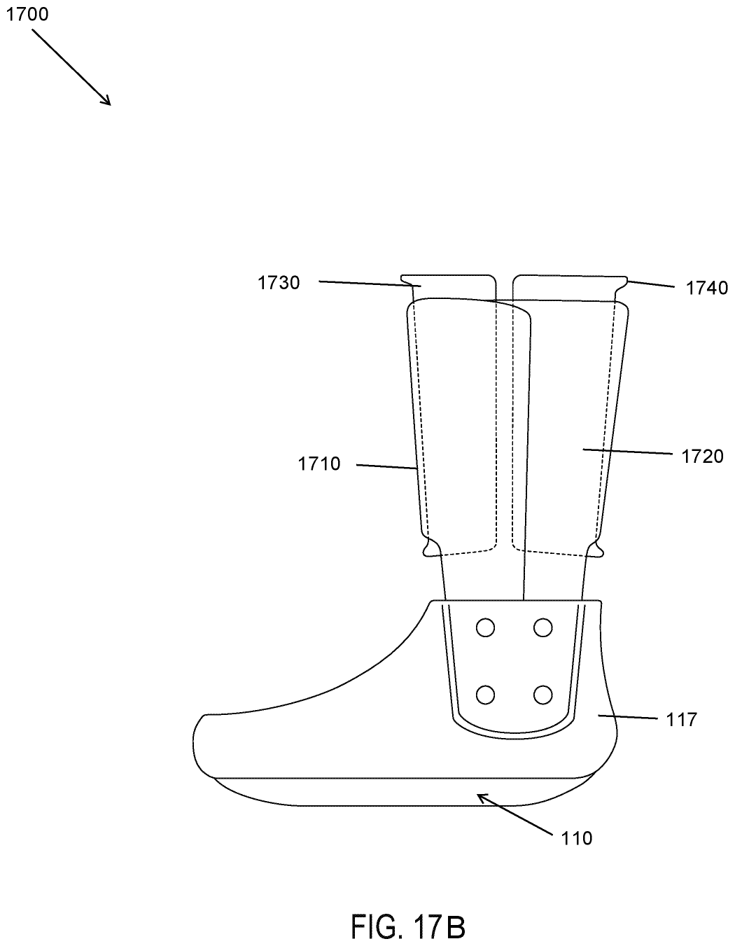

FIG. 17A depicts a foot suspension system 100 having a front vertical bar 1710 and a rear vertical bar 1720 wrapped around a front sleeve portion 1730 and a rear sleeve portion 1740. In addition to elevating the sleeve 130, the vertical bar 120 may apply a force against the sleeve 130 to secure the user leg and to ensure the sleeve 130 remains attached to the user leg or user ankle. The vertical bar 120 may be coupled to a connection point on the boot 110.

The front vertical bar 1710 and the rear vertical bar 1720 may apply a compressive force onto the sleeve 130 through its connection to the boot 110. For example, the front vertical bar 1710 and the rear vertical bar 1720 may apply an inward force on the sleeve 130 by maintaining a taut connection to the rear area 117 of the boot 110. The front vertical bar 1710 may be configured to cover a front sleeve portion 1730. Similarly, the rear vertical bar 1720 may be configured to cover a rear sleeve portion 1740. The front vertical bar 1710 and rear vertical bar 1720 may be inserted into a connection point at the rear area 117 of the boot 110. The front vertical bar 1710 and rear vertical bar 1720 may maintain a compressive inward force on the sleeve 130 through the taut connection of the front vertical bar 1710 and rear vertical bar 1720 at the rear area 117 of the boot 110. Additionally, the front vertical bar 1710 and rear vertical bar 1720 may be configured to cover a front ankle area and a back ankle area of the user to apply a compressive force against the user ankle.

The sleeve 130 may comprise a front sleeve portion 1730 and a rear sleeve portion 1740. For example, the front sleeve portion 1730 may be configured to interface with the front vertical bar 1710, and the rear sleeve portion 1740 may be configured to interface with the rear vertical bar 1720. Both the front sleeve portion 1730 and the rear sleeve portion 1740 may be configured to be held together by the compressive force of the front vertical bar 1710 and the rear vertical bar 1720. The front sleeve portion 1730 and the rear sleeve portion 1740 may include inner foam layers configured to provide comfort to the user as well as conformation to the user leg, and/or outer rigid layers configured to provide additional structural support.

In some embodiments, the front vertical bar 1710 and the rear vertical bar 1720 may apply a compressive force onto the sleeve 130 through an ankle cuff. The ankle cuff may be configured to attach to the front side or the rear side of the boot 110. More specifically, the ankle cuff may be configured to couple at a point approximately between an ankle region and a calf region of the user lower leg. The boot 110 may include an ankle cuff configured to interface with at least a portion of the front inner sleeve as well as the rear area 117 of the boot 110. The ankle cuff may be configured to cover a portion of the user's foot and ankle. The ankle cuff may provide additional protection and/or support to the user's leg. In some embodiments, the front portion and the rear portion of the sleeve 130 may be secured to the leg using an ankle cuff compressed against a back lip of the boot 110. The ankle cuff may attach using a fastener, such as snaps, buttons, hooks and loops, ties, and/or the like. The ankle cuff may be positioned around the front portion of the sleeve 130 and may be configured to hold the front sleeve portion 1730 and the rear sleeve portion 1740 in position on the user leg

FIG. 17B depicts a foot suspension system 1700 of FIG. 17A having a front vertical bar 1710 and a rear vertical bar 1720 wrapped around a front sleeve portion 1730 and a rear sleeve portion 1740.

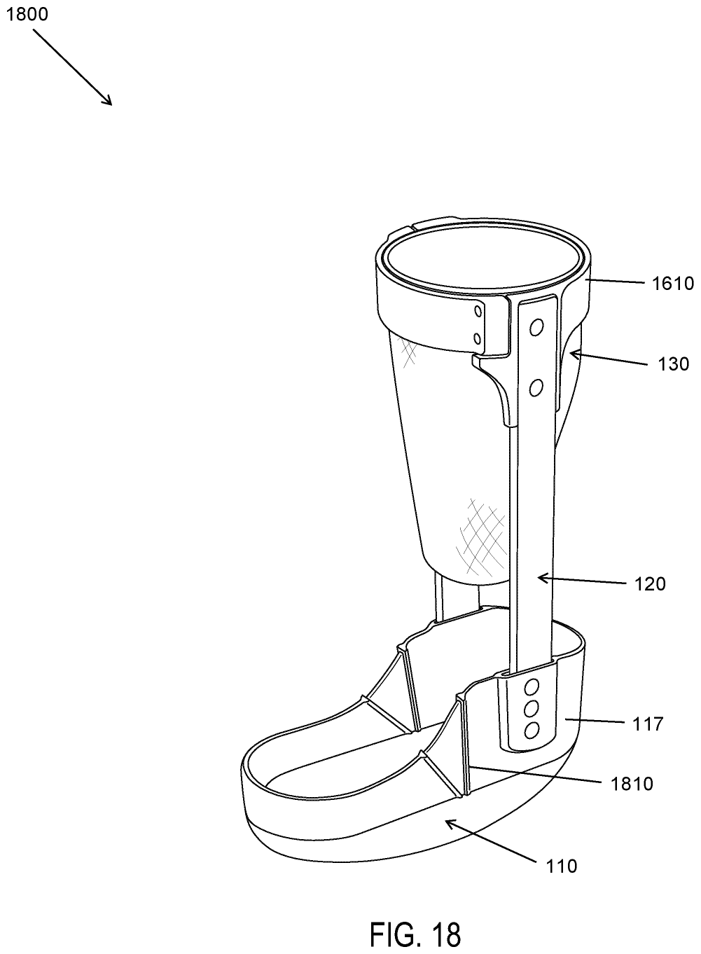

FIG. 18 depicts a side view of a foot suspension system 100 having a boot sidewall 1810 configured to flex at a midpoint. The boot sidewall 1810 may be configured to flex during a user heel strike and a user push off. The boot sidewall 1810 flexes at a midpoint in response to a movement by the sleeve 130 with respect to the boot 110. The flex in the boot sidewall 1810 enhances comfort and a natural walk for the user.

The boot sidewall 1810 may be configured to flex inward or outward in response to a downward force on the boot 110. The boot sidewall 1810 may be configured to flex inward in response to the user weight shifting from the rear area 117 of the boot 110 to the toe area 116 of the boot 110. The boot sidewall 1810 may be configured to flex such that the rear area 117 of the boot 110 and the toe area 116 of the boot 110 are closer and angled with respect to each other in response to the user weight shifting from the rear area 117 of the boot 110 to the toe area 116 of the boot 110. The boot sidewall 1810 may return to a neutral position in response to the weight and movement of the user being stationary.

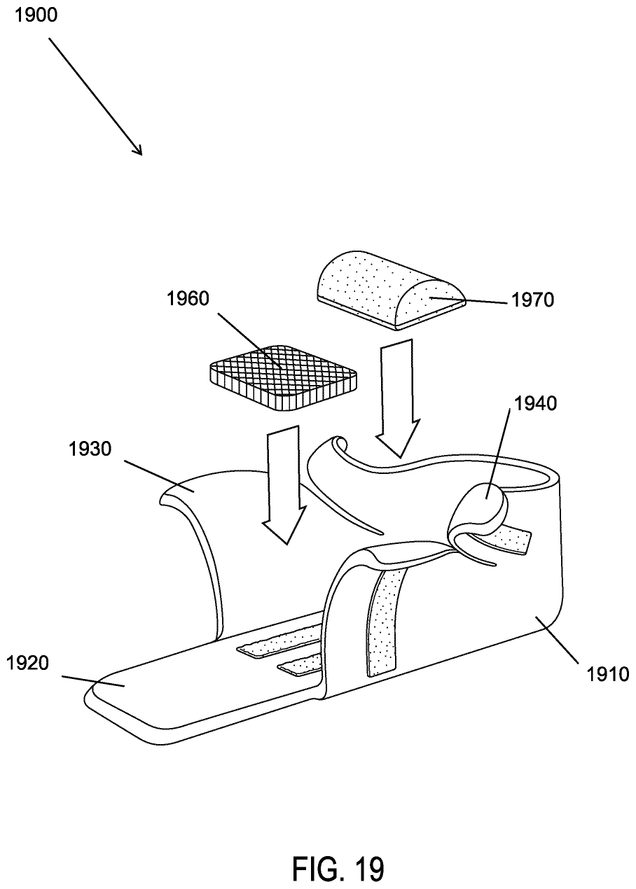

FIG. 19 depicts a perspective side view of an inner boot cover 1900. An inner boot cover 1900 may include a rear portion 1910, a front flap 1920, a side flap 1930, and an ankle flap 1940. The inner boot cover 1900 may include toe cushioning 1960 and rear cushioning 1970. The inner boot cover may provide added protection for the user foot.

The inner boot cover 1900 may be configured to fit inside the boot 110. The inner boot cover may be configured to receive the user foot and provide an additional layer of protection to the user foot. The inner boot cover is configured to detach from the user foot with various detachable flaps configured to cover various portions of the user foot. In some embodiments, the inner boot cover 1900 may include a front flap 1920 configured to expose the toes of the user. In some embodiments, the inner boot cover 1900 may include a side flap 1930, and an ankle flap to expose the dorsal and the ankle, respectively.

The toe cushioning 1960 may include detachable sections or pieces to support the toes of the user. A section or piece of the toe cushioning 1960 may be removed to prevent any pressure applied to a sore or an ulcer on the user foot. The toe cushioning 1960 may be configured to create a slope within the inner boot cover 1900. For example, the toe cushioning 1960 may be configured to create a downward slope from the rear portion 1910 to the front flap 1920. Alternatively, the toe cushioning 1960 may be configured to create an upward slope from the rear portion 1910 to the front flap 1920. The detachable sections of the toe cushioning 1960 may be removed to create a void at the portions that would otherwise contact the wound. For example, a clinician may remove the detachable sections of the toe cushioning 1960 to create a void proximate to the user wound. The remaining detachable sections of the toe cushioning 1960 may support the user foot.

The rear cushioning 1970 may include detachable sections or pieces to support the heel of the user. A section or piece of the rear cushioning 1970 may be removed to prevent any pressure applied to a sore or an ulcer on the user foot. The rear cushioning 1970 may have a dome shape such that pressure is only applied to a certain portion of the user foot. The dome shape of the rear cushioning 1970 may improve the transition from no weight on the user foot to some weight on the user foot by only affecting an isolated portion of the user foot. The rear cushioning 1970 may be used to relieve pressure on the lateral surface next to the rear cushioning 1970 between the user foot and the bottom inner surface 115. For example, if the rear cushioning 1970 is located in the midfoot area, the forefoot and hindfoot area may be relieved of the pressure between the user foot and the bottom inner surface 115. The inner boot cover 1900 may be used independently of the toe cushioning 1960 or the rear cushioning 1970 for indoor or outdoor use. Alternatively, the inner boot cover 1900 may be used in conjunction with the toe cushioning 1960 or the rear cushioning 1970 for indoor or outdoor use.

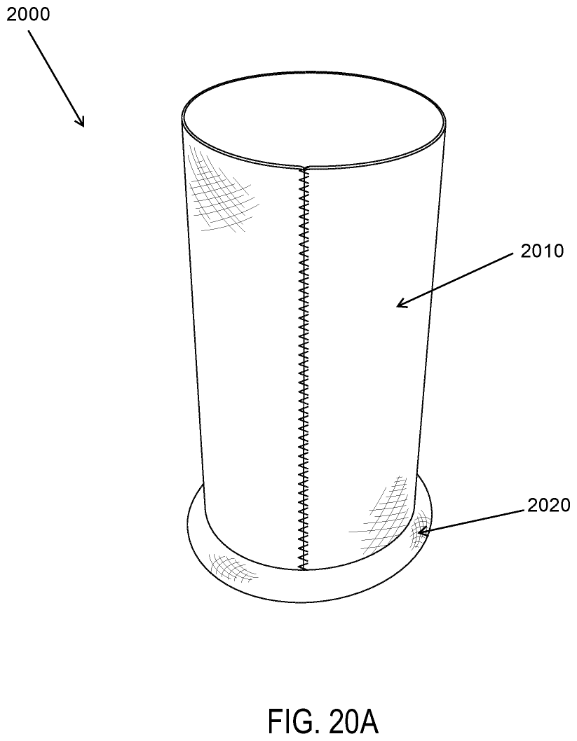

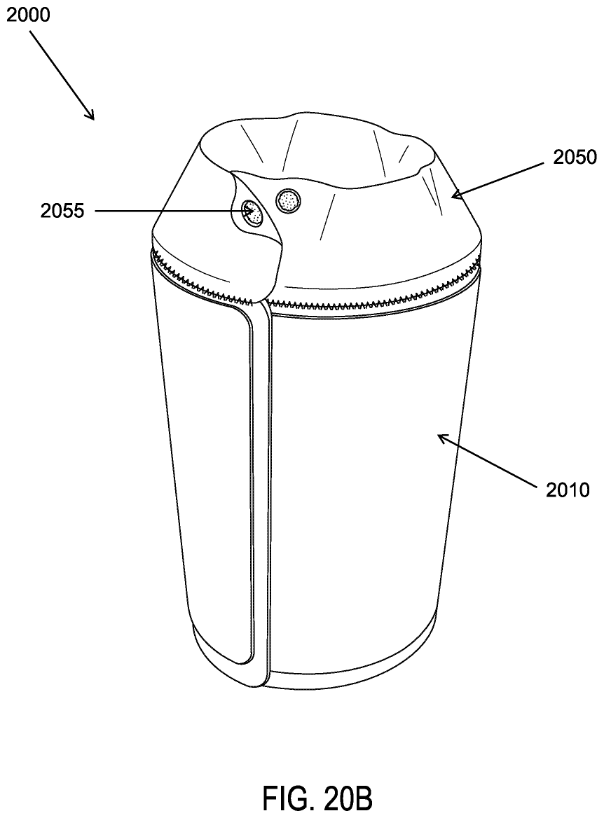

FIG. 20A depicts an inner sleeve 2000 having a slip guard 2020. The inner sleeve 2000 may be wrapped around the user calf, user ankle, or the user leg. The inner sleeve 2000 may be tapered from the top end to the bottom end to conform to the shape of the user calf to maximize compression and friction. The outside surface 2010 of the inner sleeve 2000 may include a friction-inducing material, such as a resin, a rubber, a microfiber, a polymer fiber, a leather, an adhesive, and/or the like for preventing slippage between the sleeve 130 and the lower appendage of the user. The outside surface 2010 may include a corrugated surface, a hook-and-loop surface, or an abrasive surface to prevent slippage between the sleeve 130 and the user leg. For example, the outside surface 2010 may include loops that couple to hooks at the sleeve 130 to prevent slippage between the sleeve 130 from sliding down against the inner sleeve 2000.

In some embodiments, the inner sleeve 2000 may include a slip guard 2020 at a bottom portion of the inner sleeve 2000. The slip guard 2020 may be configured to interface with a bottom portion of the sleeve 130 for increasing the likelihood that the sleeve 130 is secured to the user leg during a push off. The slip guard 2020 may be configured to prevent the user leg from slipping out of the sleeve 130 during a push off. For example, the slip guard 2020 may support the sleeve 130 from the bottom as the user steps off, preventing any downward slippage of the sleeve 130 as gravity acts on the foot suspension system 100. In some embodiments, the slip guard 2020 may include a ring that extends around the bottom portion of the sleeve 130. The slip guard 2020 may suspend the sleeve 130 during the step off until the user shifts weight or force to the foot suspension system 100.

FIG. 20B depicts a perspective side view of an inner sleeve 2000 having a cinching slip guard 2050. The inner sleeve 2000 may be wrapped around the user calf, user ankle, or the user leg. The inner sleeve 2000 may be tapered from the top end to the bottom end to conform to the shape of the user calf to maximize compression and friction. The outside surface 2010 of the inner sleeve 2000 may include a friction-inducing material, such as a resin, a rubber, a microfiber, a polymer fiber, a leather, an adhesive, and/or the like for preventing slippage between the sleeve 130 and the lower appendage of the user. The outside surface 2010 may include a corrugated surface, a hook-and-loop surface, or an abrasive surface to prevent slippage between the sleeve 130 and the user leg. For example, the outside surface 2010 may include loops that couple to hooks at the sleeve 130 to prevent slippage between the sleeve 130 from sliding down against the inner sleeve 2000.

The cinching slip guard 2050 may be configured to wrap around a portion of the lower appendage of the user. The cinching slip guard 2050 may be configured to be detachable from the sleeve 130. The cinching slip guard 2050 may be configured to prevent the inner sleeve 2000 from slipping down the user leg during heel strike. For example, the cinching slip guard 2050 may secure the inner sleeve 2000 to the user leg, preventing any downward slippage of the inner sleeve 2000 as gravity acts on the inner sleeve 2000. In some embodiments, the cinching slip guard 2050 may be a loose material that is configured to wrap around a portion of the leg of the user. The loose material may be configured to couple at an end. For example, the loose material may be configured to couple using a coupling feature 2055, such as a snap-on button, to couple the two ends of the loose material. In some embodiments, the loose material may be configured to cinch tight by pulling on a string or an elastic.

The cinching slip guard 2050 may be configured to connect at a portion of the lower appendage above the calf where the leg may be relatively thinner than the calf. In some embodiments, the cinching slip guard 2050 may be held into place by a wider circumference portion of the calf below the cinching slip guard 2050. The cinching slip guard 2050 may be situated at a top portion of the inner sleeve 2000. The cinching slip guard 2050 may suspend the inner sleeve 2000 during the heel strike until the user reduces force on the foot suspension system 100.

The terminology used herein is for the purpose of describing particular embodiments only and is not intended to be limiting of the embodiments. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” may be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description may be used in conjunction with other devices which enhance the interface between the user and the foot suspension system 100. For example, the implementations set forth may be configured to communicatively couple to a wireless monitoring component to determine the downward force on the foot suspension system 100. In another example, the foot suspension system 100 may communicatively couple to a sensor configured to measure the temperature and humidity inside the boot 110. The foot suspension system 100 may include a feedback system to notify the user when a threshold force is applied to the boot 110 or the boot 110 exceeds a threshold temperature. For example, the boot 110 may provide a signal to a speaker or a mobile device when a force detected by the sensor exceeds a threshold.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations may be provided in addition to those set forth herein. For example, the implementations described above may be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

While the foregoing is directed to implementations of the present disclosure, other and further implementations of the disclosure may be devised without departing from the basic scope thereof, and the scope thereof is determined by the claims that follow.

1. A foot suspension system configured to offload weight from a foot of a user, the foot suspension system comprising:
a boot shaped to receive at least a part of the foot of the user, the boot including a bottom inner surface;
a sleeve configured to secure to a lower appendage of the user to assist with suspending at least the part of the foot above the bottom inner surface of the boot;
at least one vertical bar including a first end and a second end, the first end of the at least one vertical bar positioned adjacent to the boot and the second end of the at least one vertical bar positioned adjacent to the sleeve, the at least one vertical bar configured to support the sleeve above the boot thereby assisting with suspending at least the part of the foot above the bottom inner surface of the boot; and
at least one rotatable pivot rotatably coupling the first end of the at least one vertical bar to the boot, the rotatable pivot allowing pivoting of the at least one vertical bar and the sleeve relative to the boot.

2. The foot suspension system of claim 1, wherein the rotatable pivot further comprises:
a dampening mechanism configured to dampen the pivoting of the at least one vertical bar and the sleeve relative to the boot,
wherein the dampening mechanism includes at least one of a magnet, an elastomeric material, a spring, a wafer spring, a honeycomb insert, and an in-mold spring.

3. The foot suspension system of claim 2, the rotatable pivot further comprising:
an engagement feature coupled to the first end of the at least one vertical bar and configured to compress the dampening mechanism as the at least one vertical bar pivots relative to the boot.

4. The foot suspension system of claim 2, wherein the rotatable pivot further comprises:
a radial protrusion feature configured to be inserted into a cutout in the dampening mechanism, the radial protrusion feature configured to secure the dampening mechanism in place at the rotatable pivot.

5. The foot suspension system of claim 4, wherein the radial protrusion feature is fixed by a plurality of teeth in a circular aperture at a rear area of the boot.

6. The foot suspension system of claim 4, wherein the dampening mechanism includes a compressible insert configured to be fixed in place by at least the radial protrusion feature in a circular aperture.

7. The foot suspension system of claim 6, the rotatable pivot further comprising:
an engagement feature coupled to the first end of the at least one vertical bar and configured to secure the compressible insert in place and configured to compress the compressible insert as the at least one vertical bar pivots relative to the boot.

8. The foot suspension system of claim 7, wherein the engagement feature is rotatable to the boot thereby compressing the compressible insert.

9. The foot suspension system of claim 6, wherein the cutout is in the compressible insert and the cutout is offset from a center of the compressible insert thereby enabling a greater dampening in a forward pivoting direction of the at least one vertical bar than a backward pivoting direction of the at least one vertical bar.

10. The foot suspension system of claim 6, wherein the cutout is in the compressible insert and the cutout is offset from a center of the compressible insert thereby enabling a greater dampening in a backward pivoting direction of the at least one vertical bar than a forward pivoting direction of the at least one vertical bar.

11. The foot suspension system of claim 7, further comprising:
a first gear including a plurality of teeth extending around a circumference of the first gear; and
a second gear configured to couple to the plurality of teeth of the first gear, the second gear configured to rotate the first gear for adjusting an angle of the at least one vertical bar with respect to the boot.

12. The foot suspension system of claim 11, wherein the first gear includes the radial protrusion feature and wherein the second gear is a worm gear having an exposed end configured to rotate the worm gear.

13-20. (canceled)

21. A foot suspension system configured to offload weight from a foot of a user, the foot suspension system comprising:
a boot having a bottom inner surface and shaped to receive at least a part of the foot of the user;
a sleeve configured to secure to a lower appendage of the user to assist with suspending at least the part of the foot above the bottom inner surface of the boot; and
at least one vertical bar including a first end and a second end, the first end of the at least one vertical bar positioned adjacent to the boot and the second end of the at least one vertical bar positioned adjacent to the sleeve, the at least one vertical bar configured to support the sleeve above the boot thereby assisting with suspending at least the part of the foot above the bottom inner surface of the boot.

22. The foot suspension system of claim 21, wherein the at least one vertical bar is adjustable to selectively secure the sleeve at more than one distance relative to the boot.

23. The foot suspension system of claim 21, the at least one vertical bar further comprises:
a shaft configured to adjust a height of the at least one vertical bar via a stepping mechanism.

24. The foot suspension system of claim 23, wherein the shaft includes an elongated aperture in a mid-section of the shaft through which a screw is inserted for securing the shaft to the at least one vertical bar.

25. The foot suspension system of claim 23, wherein the shaft includes a corrugated surface for immobilizing the shaft against the at least one vertical bar.

26. The foot suspension system of claim 21, further comprising:
a dorsal foot covering configured to cover a toe area of the boot and attach to a rear area of the boot, the dorsal foot covering configured to pivot to access the toe area of the boot, the dorsal foot covering configured to selectively attach to a front lip of the toe area.

27. The foot suspension system of claim 26, wherein the dorsal foot covering is configured to selectively detach from the rear area of the boot for accessing the foot of the user.

28. The foot suspension system of claim 26, further comprising:
a strap configured to wrap around the dorsal foot covering and coupled to a rotatable pivot.

29. The foot suspension system of claim 26, further comprising:
a strap configured to wrap around the dorsal foot covering and configured to attach to a backside of the boot.

30. The foot suspension system of claim 21, further comprising:
at least one sleeve anchor having an aperture for receiving the at least one vertical bar, the at least one sleeve anchor configured to selectively detach from the at least one vertical bar for decoupling the sleeve from the at least one vertical bar.

31. The foot suspension system of claim 30, wherein the at least one sleeve anchor further comprises:
a protrusion configured to be inserted an opening at the at least one vertical bar for securing the at least one sleeve anchor to the at least one vertical bar.

32. The foot suspension system of claim 31, wherein the sleeve anchor further comprises: a plurality of holes for fastening the sleeve anchor to a sleeve harness having a loop for securing the sleeve to the sleeve anchor.

33. The foot suspension system of claim 21, further comprising:
a sleeve harness configured to secure the sleeve to the at least one vertical bar, the sleeve harness including a loop configured to secure a horizontal strap wrapped around the sleeve to a sleeve anchor.

34. The foot suspension system of claim 33, wherein the sleeve harness includes at least two vertical straps for securing the horizontal strap.

35. The foot suspension system of claim 33, wherein the sleeve harness includes a clamp configured to extend around a circumference of the sleeve.

36. The foot suspension system of claim 21, wherein the at least one vertical bar wraps around a front portion of the sleeve, the at least one vertical bar configured to cover the front portion of the sleeve.

37. The foot suspension system of claim 21, wherein the at least one vertical bar wraps around a back portion of the sleeve, the at least one vertical bar configured to cover the back portion of the sleeve.

38. The foot suspension system of claim 21, wherein the boot includes a cutout for ventilation.

39. The foot suspension system of claim 21, wherein a boot sidewall flexes at a midpoint in response to a movement by the sleeve with respect to the boot.

40. The foot suspension system of claim 21, wherein the bottom inner surface is sloped downward from a rear area of the boot to a toe area of the boot.

41-50. (canceled)
